(19) United States
(12) Patent Application Publication
(10) Pub. No.: US 2019/0350885 A1
(43) Pub. Date: Nov. 21, 2019

(54) PROPHYLAXIS TREATMENT FOR ACUTE MYELOID LEUKEMIA

(71) Applicant: Indiana University Research and Technology Corporation, Indianlpolis, IN (US)

(72) Inventors: Mark R. Kelley, Zionsville, IN (US); Reuben Kapur, Zionsville, IN (US)

(21) Appl. No.: 16/480,010

(22) PCT Filed: Jan. 18, 2018
(86) PCT No.: PCT/US18/14252

§ 371 Date: Jul. 23, 2019

Related U.S. Application Data
(60) Provisional application No. 62/450,111, filed on Jan. 25, 2017.

(51) Int. Cl. A61K 31/192

(52) U.S. Cl. CPC A61K 31/192; A61K 31/475

(57) ABSTRACT

Compounds, and methods and uses of compounds, and pharmaceutical compositions thereof, are described herein for treating myeloid malignancies. In particular, compounds, and methods and uses of compounds, and pharmaceutical compositions thereof, are described herein for treating acute myeloid leukemia (AML), myeloproliferative neoplasm (MPN), and/or myelodysplastic syndrome (MDS).

### CROSS REFERENCE TO RELATED APPLICATIONS

This application claims the benefit to U.S. Provisional Patent Application No. 62/450,111, filed on Jan. 25, 2017, which is hereby incorporated by reference in its entirety.

### STATEMENT IN SUPPORT FOR FILING A SEQUENCE LISTING

A computer readable form of the Sequence Listing containing the file named “IURTC_2017-057-02_ST25.txt”, which is 10,509 bytes in size (as measured in MICROSOFT WINDOWS® EXPLORER), is provided herein and is herein incorporated by reference. This Sequence Listing consists of SEQ ID NOs:1-56.

### BACKGROUND OF THE DISCLOSURE

The present disclosure is generally directed to compounds, and methods and uses of compounds and pharmaceutical compositions thereof, for slowing and/or preventing the progression and/or onset of myeloid malignancies, and particularly, acute myeloid leukemia (AML), myeloproliferative disease (APN), and myelodysplastic syndrome (MDS). Particularly, it has been found that subjects having particular mutations in their hematopoietic stem cells (HSCs) have an increased probability of developing AML. The present disclosure is directed to compounds, and methods and uses of compounds and pharmaceutical compositions thereof capable of slowing and/or preventing the progression and/or onset of AML in these subjects.

Myeloid malignancies, including acute myeloid leukemia (AML), myeloproliferative neoplasia (MPN) and myelodysplastic syndromes (MDS), are clonal blood disorders. A hematopoietic stem and progenitor cell (HSPC) with mutation(s) in AML-related genes such as Tet Methylcytosine Dioxygenase 2 (TET2), DNA Methyltransferase 3 Alpha (DNMT3A) and FMS-like tyrosine kinase 3 (internal tandem duplication) (FLT3-ITD) represents what is commonly defined as a pre-leukemic HSPC (this kind of pre-leukemic HSPC is also referred to as pre-leukemic stem cell (LSC)). The selection and expansion of pre-LSC clones precede the incidence/development of AML diseases. Additionally, pre-LSCs can transform into LSCs through serial acquisition of additional somatic mutations over time and contribute to the development of full blown AML. What is unclear is the nature of environmental signals that might contribute to the “switch” from a pre-LSC state to a LSC state.

Mouse models harboring a humanized Flt3-ITD knock-in allele or carrying loss of function alleles of Tet2 or Dnmt3a manifest an expanded HSPC pool, including a hematopoietic stem cell (HSC)-enriched fraction defined by cell surface markers Lineage-/Sca-1+/c-Kit+(LSK) at a younger age. Some of these genetically-modified mice go on to develop chronic myeloid leukemia (CML) or MPN with modest penetration at an older age. However, the majority of the pre-leukemic mutations on their own seem to be insufficient to cause AML in mice, suggesting that a single mutation among the above described mutations just define a pre-leukemic condition and perhaps additional cooperating mutations in the genome (intrinsic factors) and/or environmental/microenviromental drivers (extrinsic factors) are necessary to provide a more effective selection advantage for pre-LSCs to LSCs leading to the development of full blown leukemia.

Inflammation has been linked to tumor induction and transformation in solid tissues and has recently been speculated as an enabling characteristic of cancer and its malignancies. Inflammation caused by environmental exposure, infection, autoimmunity, or ageing may result in mutations and genomic instability in somatic cells as well as in reprogramming of the tumor microenvironment (i.e., through regulating angiogenesis and expression of cytokines and chemokines). Considering that both innate and adaptive immune cells are generated from HSPCs and are involved in regulating local as well as whole-body inflammatory processes, the relationship between inflammation and hematopoietic malignancies is more complex and requires careful examination. While impact of inflammatory stress on normal HSPCs has gained attention recently, little is known about how pre-leukemic HSPCs respond to inflammation. Because HSPCs of adulthood reside in bone marrow and are surrounded by mature immune cells, the inflammatory microenvironment is likely to impact the growth and self-renewal of HSPCs in part by producing pro-inflammatory cytokines and chemokines. In support of this hypothesis are epidemiologic studies demonstrating that chronic inflammation may act as a trigger for AML development.

A recent report shows that loss of Notch signaling in the HSPC niche activates nuclear factor κB (NFκB) signaling, which in turn promotes generation of cytokine/chemokines and modulates a lethal MPN-like phenotype in mice.

Based on the foregoing, it would be beneficial to more fully understand the progression of pre-leukemic stem cells to AML. Further, it would be advantageous to provide a means for reducing and/or preventing inflammation, the production of inflammatory cytokines, and pre-leukemic stem cell generation in subjects having certain mutations in their HSPCs such to prolong or prevent the progression of myeloid malignancies, such as AML, in these subjects.

### BRIEF SUMMARY OF THE DISCLOSURE

It has been found herein that TET2-deficient pre-leukemic HSPCs have elevated NFκB/IL-6 signaling levels and maintain their regenerative advantage in primary and secondary transplantation assays, significantly outperforming wild type controls. It has further been discovered herein that compounds such as

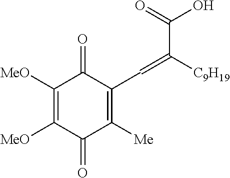

-(5-(2,3-dimethoxy-6-methyl 1,4-benzoquinoyl)]-2-nonyl-2-propenoic acid (APX3330)), and analogues thereof, can be used to provide an anti-inflammation benefit in subjects having mutations in their HSPCs, thus prolonging and/or preventing the progression of myeloid malignancies (e.g., AML, MPN and MDS) in these subjects.

Without being bound by theory, it is believed herein that, pharmacologically, an anti-inflammation drug such as APX3330 can effectively repress LPS-induced emergency granulopoiesis and LSK expansion. More importantly, APX3330 is also shown to alter the white blood cell (WBC) and red blood cell (RBC) count in aged naïve Tet2-KO mice, indicating it indeed can offer an anti-inflammation effect for the preleukemic mice. These results demonstrate that TET2-deficient bone marrow cells have distinguished tissue-repair capability in response to inflammation stress. These findings further suggest that long term TET2-deficient pre-LSCs are powered with selection advantages in clonal evolution and myeloid leukemogenesis upon stress conditions (even just aging-induced inflammation). Such intrinsic growth advantage of TET2-deficient pre-LSCs likely relies on elevated NFκB and IL-6 signaling in both mature (supplying IL-6) and immature cells (supplying and responding to IL-6) in bone marrow.

In the present disclosure, it was further found that TET2-deficient bone marrow cells have advantages in response to acute inflammation by faster emergency granulopoiesis and better repopulation capability.

Accordingly, in one aspect, the present disclosure is directed to a method of slowing the progression of a myeloid malignancy in a subject in need thereof. The method comprises administering an effective amount of 5-(2,3-dimethoxy-6-methyl 1,4-benzoquinoyl)]-2-nonyl-2-propenoic acid (APX3330)) or a pharmaceutically acceptable salt or solvate thereof.

In another aspect, the present disclosure is directed to a method of inhibiting pre-leukemic stem cell generation in a subject in need thereof. The method comprises administering an effective amount of 5-(2,3-dimethoxy-6-methyl 1,4-benzoquinoyl)]-2-nonyl-2-propenoic acid (APX3330)) or a pharmaceutically acceptable salt or solvate thereof.

In another aspect, the present disclosure is directed to a method of inhibiting production of inflammatory cytokines lacking tet methylcytosine dioxygenase 2 (TET2) in a subject in need thereof. The method comprises administering an effective amount of 5-(2,3-dimethoxy-6-methyl 1,4-benzoquinoyl)]-2-nonyl-2-propenoic acid (APX3330)) or a pharmaceutically acceptable salt or solvate thereof.

In yet another aspect, the present disclosure is directed to a method of repressing inflammation in a subject having at least one mutation in a hematopoietic stem cell. The method comprises administering an effective amount of 5-(2,3-dimethoxy-6-methyl 1,4-benzoquinoyl)]-2-nonyl-2-propenoic acid (APX3330)) or a pharmaceutically acceptable salt or solvate thereof.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIGS. 1A-1P depict that TET2-KO mice exhibited extended granulopoiesis and splenomegaly in response to acute inflammatory challenge. FIGS. 1A-1C and 1F-1K depict hematologic changes in the peripheral blood (PB) of LPS-treated wildtype and Tet2-KO mice (0.8 mg/kg, one dose, i.p.) over a 7-day period. Note that the frequency (Freq.) of neutrophils is significantly increased and the frequency (Freq.) of lymphocytes is significantly decreased in Tet2-KO mice compared to wildtype controls on Day 2 (FIGS. 1C & 1H). FIGS. 1D & 1E depict quantification of eosinophil (EO) and basophil (BA) counts. FIGS. 1L-1N depict changes in spleen weight during LPS-induced acute inflammation. FIG. 1O depicts the impact on mature cells in the bone marrow and spleen. FIG. 1P depicts changes in bone marrow and spleen pre- and post-LPS treatment. Data are presented as mean+s.e.m. (n=10 biological repeats per each group). Experiments were repeated at least three times. P value: * P<0.05, ** P<0.01.

FIGS. 2A-2J show enhanced recovery of HSPCs in Tet2-KO mice in response to inflammatory challenge. FIG. 2A depicts representative flow cytometry profiles showing changes in the LSK, HSC, CMP, GMP and MEP populations in LPS treated-mice on Day 1 compared to naïve mice (Day 0). Note that the LSK compartment is expanded while the CMP compartment is reduced on Day 1 (day-to-day comparison). In addition, Tet2-KO mice demonstrate higher frequency of LSKs and CMPs relative to wildtype controls. Schematic of Hematopoiesis from HSCs to mature immune cells is shown below the cytometry profiles. FIG. 2B depicts changes in bone marrow cellularity (No.) from Day 0 to Day 7 post LPS treatment. Note the acute reduction in the bone marrow cellularity on Day 1 and Day 2 and slow recovery between Day 3 and Day 7 post LPS treatment. No significant differences between wild type and Tet2-KO mice in their bone marrow cellularity were observed at any of the time points examined. FIGS. 2C-2G depict the quantification of the frequencies (Freq.) and absolute cell numbers (No.) of CMP, GMP, MEP, LSK and HSC between Day 0 and Day 7 post LPS treatment. FIG. 2H depicts the frequency (Freq.) of c-Kit+ and Sca-1+ cells in Lin-negative bone marrow cells between Day 0 and Day 7 post LPS treatment. FIG. 2I depicts expression (Expr.) levels of c-Kit and Sca-1 in Lin-negative bone marrow cells measured by mean of inflorescence intensity (MFI). FIG. 2J depicts quantification of the frequency (Freq.) and absolute cell number (No.) of CMPs, LSKs and HSCs on day 0 and day 2 post LPS treatment in juvenile 3 to 4 week old wildtype and Tet2-KO mice. Experiments were repeated at least three times. P value: * P<0.05, ** P<0.01.

FIGS. 3A-3D depict quantification of CLP, MPP and short term-HSC compartments and representative histogram plots of flow cytometry analyzed with c-Kit and Sca-t markers. Data are presented as mean+s.e.m. (n=4 biological repeats per each group). Experiments were repeated at least three times. P value: * P<0.05, ** P<0.01.

FIGS. 4A-4J show that Tet2-KO hematopoietic progenitor cells show reduced apoptosis, enhanced proliferation and DNA damage in response to acute inflammatory challenge. FIG. 4A depicts the level of apoptosis in Lin-negative cells and LSK cells post LPS treatment (Day 0 to Day 2) as assessed by Annexin-V/7-AAD flow cytometry. FIG. 4B depicts the presence of Ki-67+ proliferating cells within the Lin-negative or the LSK fraction of the bone marrow post LPS treatment (Day 0 to Day 2). FIGS. 4C & 4D depict acute changes in DNA damage within the Lin-negative and the LSK fractions of the bone marrow as assessed by γH2AX staining. The level of expression of γH2AX is indicated by MFI. FIGS. 4E-4J show the qRT-PCR analysis of representative pro-apoptotic genes (Casp1 and Bcl2111), pro-survival genes (Bcl2 and Morrbid), cell-cycle regulators-encoding genes (Ccne1 and Ccnd1) and genes encoding DNA-damage responsive regulators (53bp1 and Rad51) or DNA-repair pathway regulators (Fanca, Fancd2, Xrcc5 and Atm). Data are presented as mean+s.e.m. (n=4 biological repeats per each group). Experiments were repeated at least twice. P value: * P<0.05, ** P<0.01.

FIGS. 5A-5E depict representative flow cytometry plots of Annexin-V/7-AAD staining and histogram plots of Ki67 and γH2AX staining Data are presented as mean+s.e.m. (n=4 biological repeats per each group). Experiments were repeated at least twice. P value: * P<0.05, ** P<0.01.

FIGS. 6A-6J show that LPS-stressed Tet2-deficient bone marrow cells maintain repopulation advantage. FIG. 6A is a schematic describing primary and secondary competitive bone marrow transplantation (cBMT) assay. For primary cBMT assay, donor cells from naïve mice (Day 0, wildtype or Tet2-KO) or from LPS-treated mice (Day 1, Day 2 and Day 3 post LPS treatment, wildtype or Tet2-KO) (all are CD45.2+) were mixed equally with donor cells from naïve BoyJ mice (CD45.1+) and transplanted into irradiated recipient animals (CD45.2+/CD45.1+). For secondary cBMT assay, donor cells from primary recipients were mixed equally with donor cells from naïve BoyJ mice and transplanted into irradiated recipient animals (CD45.1+/CD45.2+). FIGS. 6B & 6C depict that Tet2-deficient bone marrow with or without LPS treatment demonstrated significantly higher engraftment of CD45.2 cells in the primary and secondary recipients compared to wildtype controls. Note that Day 2 LPS treated wildtype CD45.2 bone marrow donor cells lost their normal engraftment ability to non-treated and Day 1 LPS treated cells. FIG. 6D is a quantification of CD45.2+ donor cells in ungated bone marrow viable total cells (BM_Live), Lin-negative, LSK cells, myeloid cells, B cells and T cells. FIGS. 6E & 6F show that Tet2-deficient bone marrow donor cells induced splenomegaly, myeloid cell skewing and defective B cell development in primary recipient mice. FIGS. 6G-6I depict identical number of LSK cells were purified from wildtype and Tet2-KO mice pre- and post-LPS treatment and subjected to in vitro CFU assay (FIG. 6H) and in vivo cBMT (FIG. 6I) assay, respectively. Transplant experiments were conducted as described in (FIG. 6G). FIG. 6J depicts representative flow cytometry plots of CD45.2/CD45.1 chimerism. Quantitative analysis of chimerism in various bone marrow subsets after 16 weeks of transplantation in primary recipients. Data are presented as mean+s.e.m. (n=5 for each cBMT experiment). Primary cBMT were repeated twice. Secondary cBMT was performed once with 5 mice in each group. P value: * P<0.05, ** P<0.01.

FIGS. 7A-7G show that Tet2-KO mice show increased expression of IL-6 in serum and in various bone marrow subsets. FIG. 7A depicts increased expression of multiple cytokines and chemokines including IL-6, CCL2, CCL4 and TNFα in response to LPS in Tet2-KO mice. The average serum levels of each cytokine or chemokine in wildtype mice in naïve conditions (day 0) was defined as fold 1. Fold changes in serum cytokine/chemokine levels in pre-LPS treated Tet2-KO mice or post LPS treated mice were calculated and plotted accordingly.

FIGS. 7B & 7C depict intracellular flow cytometry analysis (ICFC) of IL-6 expression in total bone marrow cells and in Lin-negative bone morrow cells pre- and post-LPS treatment. FIG. 7D depicts expression of IL-6, TNFα, IL-1β and GM-CSF in bone marrow Lin-negative cells as assessed by flow cytometry and MFI quantification. FIG. 7E depicts QRT-PCR analysis of IL-6 and Ccl2 expression in bone marrow Lin-negative cells. FIGS. 7F & 7G depict QRT-PCR analysis of IL-6, TNFα, Ccl2, and Ccl4 expression in bone marrow Lin-negative cells derived from adult and juvenile pre- and post-LPS treated wildtype and Tet2-KO mice.

FIGS. 8A-8F depict representative IFC plots for TNFα, IL-1β and GM-CSF. Data are presented as mean+s.e.m. (n=4 biological repeats per each group). Experiments were repeated at least twice. P value: * P<0.05, ** P<0.01.

FIGS. 9A-9J show that Tet2-KO mice have increased expression of TLR4/NFκB/IL-6 pathway. FIG. 9A is a schematic describing an abbreviated form of the canonical TLR4/NFκB/IL-6 and putative IL-6/Stat1/Sca-1 pathways. Activation of TLR4 is through an exogenous ligand such as LPS (when infection available) or possibly through endogenous ligands S100A8/S100A9 (when no infection available). Repression of the pathways by APX3330 acts on the level of Ape1-NFκB or putatively on the level of Ape1-Stat3. FIG. 9B depicts increased frequencies of TLR4+ cells and increased expression of TLR4 (calculated by flow cytometry and MFI) in bone marrow cells from naïve Tet2-KO mice relative to wild type controls. FIG. 9C depicts an qRT-PCR assay performed on cells derived from wild type and Tet2-KO mice to analyze the expression for Tlr4, Ticam1, Nfkb1, Nfkbiz, Apex1, Stat1, Stat3 and Ly6a. FIGS. 9D & 9E show that a short-term treatment of APX3330 represses emergency neutrophil production in peripheral blood and maintained normal bone marrow cellularity in both wild type and Tet2-KO. In addition, APX3330 represses the expansion of LSK cells in the bone marrow both wildtype and Tet2-KO. FIG. 9F depicts increased expression of NFκB1 and phospho-Stat3 in Tet2-KO Lin-negative bone marrow cells pre- and post-LPS challenge. FIG. 9G depicts qRT-PCR analysis on Lin-cells derived from naïve wildtype and Tet2-KO mice showing the expression of Tlr4, Ticam1, Nfkb1, Nfkbiz, and Apex1 in Lin-negative bone marrow cells. FIG. 9H depicts enhanced binding of IκBζ to IL-6 and TLR4 promoter as revealed by CHIP-qPCR analysis. FIGS. 9I & 9J depict that E3330 or SHP099 treatment of Tet2-KO Lin-negative bone marrow cells corrected the enhanced colony forming ability of Tet2-KO cells in primary and secondary replating assay and rescues the enhanced binding of IκBζ and Stat3 to the Morribid promoter as revealed by CHIP-qPCR assay. Data are presented as mean+s.e.m (n=3 or 4 biological repeats per each group). Experiments were repeated at least twice. P value: * P<0.05, ** P<0.01.

FIGS. 10A-10C are representative flow cytometry plots of TLR4 and IL-6Rα labeling in various fractions of bone marrow cells. Data are presented as mean+s.e.m (n=3 or 4 biological repeats per each group). Experiments were repeated at least twice. P value: * P<0.05, ** P<0.01.

FIGS. 11A-11D show that APX3330 reverses early signs of MPN in aged Tet2-KO mice. FIG. 11A depict that aged Tet2-KO mice (6-month old) exhibit early signs of MPN as shown by splenomegaly and increased counts and frequencies (Freq.) of neutrophils in peripheral blood. FIG. 11B is a schematic describing a treatment procedure of aged Tet2-KO mice with APX3330. FIG. 11C depicts hematological parameters and spleen weight in APX3330-treated aged Tet2-KO mice. Although APX3330 failed to alter splenomegaly in aged Tet2-KO, note that both absolute numbers and percentages of neutrophils are significantly reduced compared to control while red blood cell (RBC) counts and hematocrits (HCT) are rescued to normal levels. FIG. 11D shows a model for myeloid skewing and altered HSC activity induced by Tet2 deficiency and/or inflammatory stress. Loss of Tet2 in the pre-leukemic mice maintains increased basal levels of TLR4, IL-6 and Sca-1 proteins compared to normal mice. Upon inflammatory stress, Tet2-deficient mice showed enhanced emergency granulopoiesis and hematopoiesis (myeloid skewing), in part by regulating the expression of TLR4, IL-6 and Sca-1. While wild type HSCs are susceptible to inflammatory stress, Tet2-deficient HSCs are resistant to such form of stress and maintained self-renewal and repopulating advantage compared to wildtype cells. Data in FIG. 11A are presented as mean+s.e.m. Data in FIG. 11C are presented as mean+s.d. (n=5 biological repeats per each group). Experiments were repeated at least twice. P value: * P<0.05, ** P<0.01.

FIG. 12 shows that Tet2-KO hematopoietic progenitor cells showed sustained cell survival, enhanced proliferation and DNA damage in response to acute inflammatory challenge. Particularly, FIG. 12 depicts that Stat3 binds to Morrbid locus revealed by CHIP-qPCR enrichment assay. Shown is relative enrichment in binding.

FIGS. 13A-13O show that E3330 and SHP099 treatment reversed early signs of MPN in aged Tet2-KO mice. FIGS. 13A, 13D, 13E, 13I, 13L and 13M depict alterations in PB parameters after a two-week treatment period with E3330 or SHP099 in aged Tet2-KO mice. FIG. 13A is a schematic showing the strategy for drug treatment in Tet2-KO mice pre-LPS treatment. FIGS. 13D & 13E are representative flow cytometry profiles of LSK cells post-LPS and drug treatment. FIG. 13I is a schematic showing drug treatment strategy in aged Tet2-KO mice. FIGS. 13L & 13M depict PB parameters and spleen weight changes in aged Tet2-KO mice treated for 14 days with E3330 or SHP099. FIGS. 13B & 13C depict that pre-treatment of Tet2-KO mice with E3330 or SHP099 repressed LPS-induced emergency neutrophil production and LSK cell expansion. FIGS. 13F-13H show that aged Tet2-KO mice developed splenomegaly, neutrophilia and increase serum IL-6 levels. FIGS. 13J, 13K, 13N and 13O show rescue of PB neutrophil counts, neutrophil frequency and serum IL-6 levels in aged Tet2-KO treated with E3330 or SHP099. Data are mean+s.e.m. Experiments were repeated at least twice. P value: * P<0.05, ** P<0.01.

### DETAILED DESCRIPTION

Myeloid malignancies including acute myeloid leukemia (AML), myeloproliferative neoplasia (MPN) and myelodysplastic syndromes (MDS) are clonal blood disorders. More particularly, AML is a myeloid cell cancer characterized by rapid growth and accumulation of abnormal white blood cells in bone marrow and blood. These malignant cells interfere with the normal production of red blood cells and platelets, causing anemia and pathologic bleeding. AML is caused by genetic changes, and particularly, mutations in hematopoietic stem and progenitor cells (HSPCs) that result in increased cellular growth and proliferation, and impaired maturation. A hematopoietic stem and progenitor cell (HSPC) with one or more mutations in AML-related genes such as Tet Methylcytosine Dioxygenase 2 (TET2), DNA Methyltransferase 3 Alpha (DNMT3A) and FMS-like tyrosine kinase 3 (internal tandem duplication) (FLT3-ITD) represents a pre-leukemic clone in humans (this kind of pre-conditioned HSPC is also referred to as pre-leukemic stem cells pre-LSC). The pre-LSC clones can develop into more aggressive (with advantages in selection and expansion of the clones) malignancies through a serial acquisition of additional somatic mutations over time in the cells.

In many situations, the progression of pre-LSC to full blown AML can be triggered by inflammation. For example, it has been found that AML more prominently develops in subjects with other inflammatory diseases, disorders and conditions, particularly, subjects suffering from aging, diabetes, obesity, chronic infections, smoking, arthritis, and combinations thereof.

The term “subject” is used interchangeably herein with “patient” to refer to an individual to be treated. The subject is a mammal (e.g., human, non-human primate, rat, mouse, cow, horse, pig, sheep, goat, dog, cat, etc.). The subject can be a clinical patient, a clinical trial volunteer, a companion animal, an experimental animal, etc. The subject can be suspected of having or at risk for having a condition (such as a myeloid malignancy (e.g., AML, MPN, MDS)) or be diagnosed with a condition (such as a preleukemic disorder or condition). The subject can also be suspected of having or being at risk for having a myeloid malignancy. According to one embodiment, the subject to be treated is a human.

Tet Methylcytosine Dioxygenase 2 (TET2) catalyzes the 5-hydroxylation of methylcytosine (5-mc) to 5-hydroxymethylcytosine (5-hmc) and is an essential epigenetic regulator for the human genome. TET2 was just recognized as a tumor suppressor in cancer biology less than ten years ago. Although it has been validated that TET2-deficient LSK/HSC cells have increased self-renew activity using a mouse in vivo model, it's largely unknown the underlying molecular mechanisms.

It has been found herein that, although the hematological counts in peripheral blood of both wild type and Tet2-knockout (Tet2-KO) mice return to a normal level at the late stage of a lipopolysaccharides (LPS)-induced acute inflammation challenge, the granulopoiesis (as indicated by neutrophil cell counts) and activation of hematopoietic stem and progenitor cells (HSPCs) were robustly extended during the early stage in the Tet2-KO mice. Without being bound by theory, it is believed that this dramatic difference is likely attributed to a significantly increased supply of pro-inflammatory cytokine IL-6 in the serum of preleukemic mice. Moreover, genome instability and progenitor cell survival rates were observed at a higher scale in Tet2-KO during the acute inflammatory stress. Functionally, competitive transplantation assays further confirmed that LPS-stressed Tet2-deficient bone marrow cells maintain repopulation advantages against the wild type donor controls in long-term engraftment Finally, Tet2-KO mice maintained elevated TLR4/NFκB signaling in naïve condition or during LPS stress.

Based on the foregoing, in one embodiment of the present disclosure, there is provided a method of slowing the progress of a myeloid malignancy in a subject in need thereof. As used herein, “slowing the progression” or “slowing the progress” refers to delaying the onset, preventing or slowing the spread or stage of the malignancy, and/or reducing complications of the malignancy as compared to a patient not administered 5-(2,3-dimethoxy-6-methyl 1,4-benzoquinoyl)]-2-nonyl-2-propenoic acid (APX3330)) or a pharmaceutically acceptable salt or solvate thereof. In one embodiment, the myeloid malignancy is acute myeloid leukemia (AML). In another embodiment, the myeloid malignancy is myeloproliferative neoplasia (MPN). In yet another embodiment, the myeloid malignancy is myelodysplastic syndrome (MDS).

In another embodiment, the present disclosure provides a method of inhibiting pre-leukemic stem cell generation in a subject in need thereof.

In yet another embodiment, the present disclosure provides a method of inhibiting production of inflammatory cytokines lacking tet methylcytosine dioxygenase 2 (TET2) in a subject in need thereof.

In another embodiment, the present disclosure provides a method of repressing inflammation in a subject having at least one mutation in a hematopoietic stem cell. Generally, the inflammation will be statistically decreased using the methods described herein.

Generally, in the methods of the present disclosure, an effective amount of 5-(2,3-dimethoxy-6-methyl 1,4-benzoquinoyl)]-2-nonyl-2-propenoic acid (APX3330)) or a pharmaceutically acceptable salt or solvate thereof is administered to a subject in need thereof. It has been found herein that APX3330 partially reversed the extended inflammation phenotype in Tet2-deficient mice. More particularly, as shown in the Examples below, APX3330 effectively repressed LPS-induced emergency granulopoiesi and LSK expansion. More importantly, APX3330 was shown to alter the white blood cell (WBC) and red blood cell (RBC) count in aged naïve Tet2-KO mice, indicating it indeed offered an anti-inflammation effect for the preleukemic mice.

3-[(5-(2,3-dimethoxy-6-methyl1,4-benzoquinoyl)]-2-nonyl-2-proprionic acid (hereinafter “E3330” or “3330” or “APX3330”) selectively inhibits the redox function of APE1/Ref-1. Apurinic/apyrimidinic endonuclease 1 redox factor 1 (APE1/Ref-1) is a multifunctional protein that has recently been found to be essential in activating oncogenic transcription factors. Further information on APX3330 may be found in Abe et al., U.S. Pat. No. 5,210,239, incorporated herein by reference to the extent it is consistent herewith.

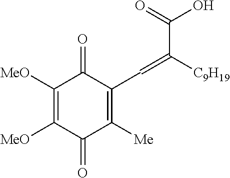

-(5-(2,3-dimethoxy-6-methyl 1,4-benzoquinoyl)]-2-nonyl-2-propenoic acid (APX3330))

Where subject applications are contemplated, particularly in humans, it will be necessary to prepare pharmaceutical compositions including APX3330 in a form appropriate for the intended application. Generally, this will entail preparing compositions that are essentially free of impurities that could be harmful to a subject.

The compound (i.e., APX3330) and compositions can be administered orally, intravenously, intramuscularly, intrapleurally or intraperitoneally at doses based on the body weight and degree of disease progression of the subject, and may be given in one, two, three or even four daily administrations. For example, in some embodiments, APX3330 is administered in amounts ranging from about 10 mg/kg to about 75 mg/kg, including from about 15 mg/kg to about 50 mg/kg, and including about 25 mg/kg.

One will generally desire to employ appropriate salts and buffers to render the compounds stable and allow for uptake by target cells. Aqueous compositions of the present disclosure comprise an effective amount of the compound, dissolved or dispersed in a pharmaceutically acceptable carrier or aqueous medium. Such compositions also are referred to as innocuous. The phrase pharmaceutically or pharmacologically acceptable refers to molecular entities and compositions that do not produce adverse, allergic, or other untoward reactions when administered to a subject. As used herein, pharmaceutically acceptable carrier includes any and all solvents, dispersion media, coatings, antibacterial and antifungal agents, isotonic and absorption delaying agents and the like. The use of such media and agents for pharmaceutically active sub-stances is well known in the art. Supplementary active ingredients also can be incorporated into the compositions.

Compositions for use in the present disclosure may include classic pharmaceutical preparations. Administration of these compositions according to the present disclosure will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, as described herein.

For example, the compounds can be formulated with common excipients, diluents, or carriers, and formed into tablets, capsules, suspensions, powders, and the like. Examples of excipients, diluents, and carriers that are suitable for such formulations include the following: fillers and extenders such as starch, sugars, mannitol, and silicic derivatives; binding agents such as carboxymethyl cellulose and other cellulose derivatives, alginates, gelatin, and polyvinyl pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols.

APX3330 may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In some particularly suitable embodiments, the form is sterile and is fluid to the extent that easy syringability exists. It can be stable under the conditions of manufacture and storage and can be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

For oral administration, compounds of the present disclosure may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including gels, pastes, powders and slurries. The active ingredient may be added in a therapeutically effective amount to a paste dentifrice that may include water, binders, abrasives, flavoring agents, foaming agents, and humectants.

The compositions for use in the present disclosure may be formulated in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, general safety and purity standards as required by FDA and foreign counterpart agencies.

The methods described herein can further include administering one or more antileukemia chemotherapeutic agent or one or more antileukemia enzyme inhibitor, or a combination thereof with APX3330. For example, one or more antileukemia chemotherapeutic agent selected from the group consisting of dexamethasone, vincristine, doxorubicin, and methotrexate can be administered with APX3330. In other embodiments of the present disclosure, the methods can further include administering an anti-inflammatory with APX3330, for examples, anti-inflammatory agents such as anti-IL6 antibodies and/or NFκB inhibitors.

The methods described herein can further include administering one or more additional therapeutic agents. Exemplary additional therapeutic agents include an inhibitor of signal transducer and activator of transcription 3 (STATS) (e.g., 6-(4-amino-4-methyl-1-piperidinyl)-3-(2,3-dichlorophenyl)-2-pyrazinamine (SHP099); 2-Hydroxy-4-(((4-methylphenyl)sulfonyloxy)acetyl)amino)-benzoic acid/S3I-201, 6-Nitrobenzo[b]thiophene-1,1-dioxide/stattic, OCHROMYCINONE, 4-(N-(4-Cyclohexylbenzyl)-2-(2,3,4,5,6-pentafluoro-N-methylphenylsulfonamido)acetamido)-2-hydroxybenzoic acid; napabucasin). In one particular embodiment, the methods include administering APX3330 with SHP099.

The following examples and procedures further illustrate specific embodiments of the invention; however, the following illustrative examples should not be interpreted in any way to limit the invention.

### EXAMPLES

### Example 1

In this Example, it was analyzed whether TET2-deficient HSPC maintain a leukemia-promoting advantage during physiological stress by examining how TET2-KO mice respond to acute inflammation.

### Materials and Methods

Mice, LPS treatment and peripheral blood analysis. All mice were bred and maintained under specified pathogen-free (SPF) conditions at an animal facility at Indiana University School of Medicine. Experiments with mice were approved by the Institutional Animal Care and Use Committee (IACUC) of Indiana University School of Medicine. Tet2-knockout mice (Tet2^{-/-}, or Tet2-KO, CD45.2) is on C57BL/6 genetic background and has been previously described in Li et al., Blood 118, 4509-4518 (2011). Normal C57BL/6 (wild type, CD45.2) mice were purchased from The Jackson Laboratory and used as controls for all experiments. Whenever possible littermates were used as controls for all experiments.

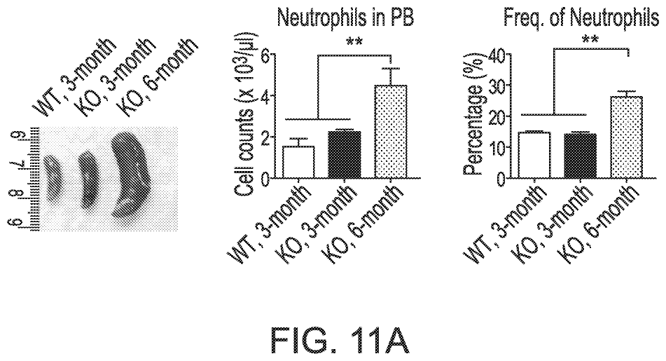

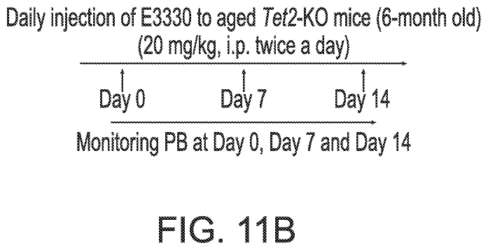

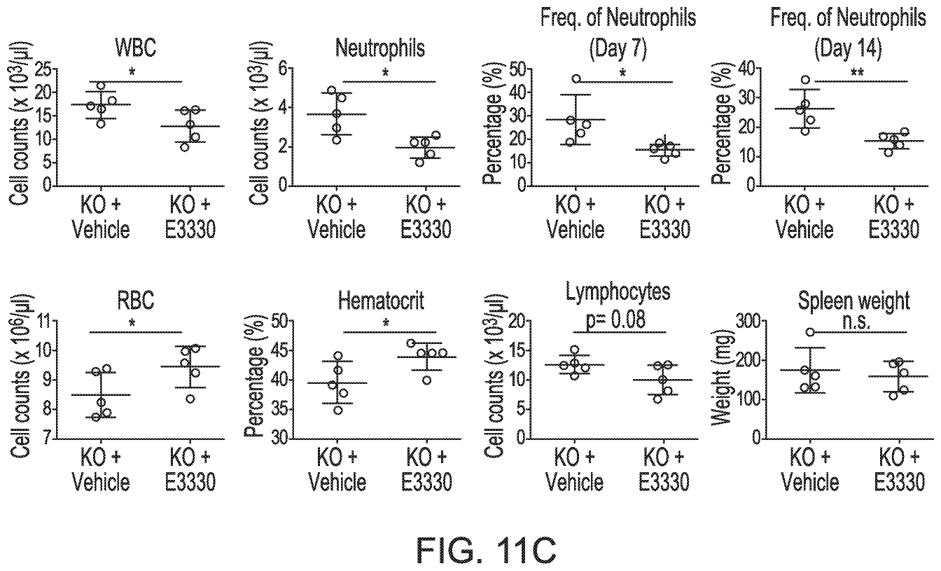

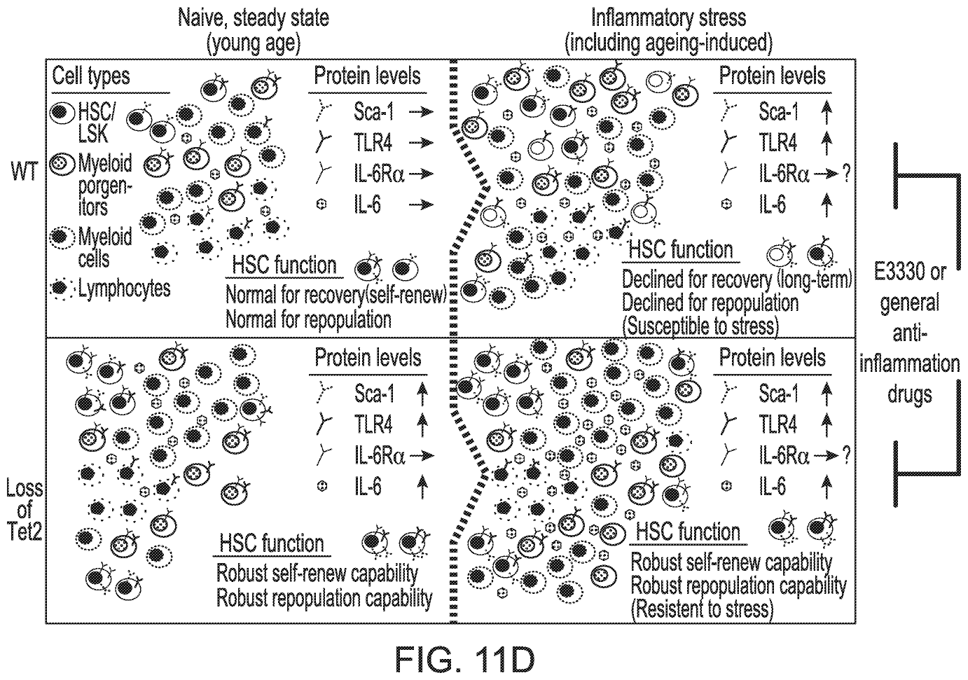

Lipopolysaccharide (LPS) was purchased from Sigma (Cat # L8643) and dissolved in sterile phosphate-buffered saline (PBS) prior to being given to the mice for one dose only (0.8 mg/kg, i.p.). APX3330 (also referred to herein as E3330) was dissolved in Cremophor:EtOH (1:1) (Cremophor were purchased from Sigma, Cat # C5135) for making solution stock and then diluted in PBS prior to be used for pre-LPS treatment or post-LPS treatment (20 mg/kg, twice a day, i.p.). SHP099 (provided by Norvartis), was dissolved in 0.5% Methylcellulose (Sigma, Cat# M0262) and 0.1% Tween-80 (Fisher Scientific, Cat #BP338-500) and fed to animals by gavage (daily, 50 mg/kg). Male and female mice between 3-4 weeks (juvenile mice) or 8-16 weeks (adult mice) of age were used for LPS or LPS plus APX3330 experiments. Age and sex matched mice were always used as naïve (Day 0) controls. Aged Tet2-KO mice (male or female, 6-8 months of age) were used for APX3330 or SHP099 treatment in FIGS. 11A-11D.

Hematological analysis on peripheral blood (PB, from tail-bleeding) was run by an automated cell counter machine (Drew Hemavet 950). Total bone marrow (BM) cells were harvested from two femurs and two tibias of mice and filtered on 50-μm sterile filters. BM cells were always kept on ice or in refrigeration and stored in sterile blocking buffer containing 2% rat-serum prior to analysis. BM cellularity (viable cell counts) was analyzed by an automated cell counter (Beckman the Vi-CELL™ Cell Counter for Cell Viability Analyzer).

Flow cytometry. Non-lysed BM cells were used for analysis of erythroid lineage and progenitor cells (Ter119 and CD71 staining). Remaining flow cytometry analysis was performed on lysed bone marrow cells (Lysis Buffer, BD, Cat #555899). Antibodies against Ter119, Mac1, Gr1, B220, CD3, CD4 and CD8 were used for mature cells labeling (Linage labeling). Progenitor cells were labeled and analyzed by indicated markers. Antibody-labeled BM cells were run on a BD FACS-CANTO II machine with a two-laser and six-filter configuration. The properly compensated flow data were analyzed by Flow Jo software (V10.2). Events plotting, calculation of frequency and mean of fluorescence intensity (MFI), and histogram overlaying were analyzed by Flow Jo software. A full list of staining schemes, gating strategies and antibodies is provided in Table 1.

TABLE 1

| | | SEQ | |
|---|---|---|---|
| | | ID | |
| qRT-PCT primers used in FIGS. 11A-11D | | NO | Source |
| Casp1, | AGTCCTGGAAATGTGCCATC | 1 | https://mouseprimerdepot.nci.nih. |
| Forward | | | gov/ |
| Casp1, | TCAGCTCCATCAGCTGAAAC | 2 | |
| Reverse | | | |
| Bcl2111, | CGCAGATCTTCAGGTTCCTC | 3 | Kotzin, J. J. et al., Nature, 2016 |
| Forward | | | |
| Bcl2, | ACAAACCCCAAGTCCTCCTT | 4 | |
| Forward | | | |
| Bcl2, | GGTCTTCAGAGACAGCCAGG | 5 | https://mouseprimerdepot.nci.nih. |
| Forward | | | gov/ |
| Bcl2, | GATCCAGGATAACGGAGGCT | 6 | |
| Reverse | | | |
| Morrbid, | TCTGAGAATGAGGGGACTGG | 7 | Kotzin, J. J. et al., Nature, 2016 |
| Forward | | | |
| Morrbid | TGTGCTGTGAAGATCCCAAG | 8 | |
| Reverse, | | | |
| Ccne1, | GCAGCGAGCAGGAGACAGA | 9 | https://mouseprimerdepot.nci.nih. |
| Forward | | | gov/ |
| Ccne1, | GCTGCTTCCACACCACTGTCTT | 10 | |
| Reverse | | | |
| Ccnd1, | TGTTACTTGTAGCGGCCTGTTG | 11 | https://mouseprimerdepot.nci.nih. |
| Forward | | | gov/ |
| Ccnd1 | CCGGAGACTCAGAGCAAATCC | 12 | |
| Reverse, | | | |
| 53bp1, | TACAGCCCGGTAAAGGTATCC | 13 | Flach, J. et al., Nature, 2014 |
| Forward | AT | | |
| 53bp1, | CTGGACGGCCGGTCTTC | 14 | |
| Reverse | | | |
| Rad51, | AAGTTTTGGTCCACAGCCTAT | | |
| Forward | TT | 15 | Flach, J. et al., Nature, 2014 |
| Rad51, | CGGTGCATAAGCAACAGCC | 16 | |
| Reverse | | | |
| Fanca, | GCCTCCGAAAAACTTGGACC | 17 | Walter, D. et al., Nature, 2015 |
| Forward | | | |
| Fanca, | GGCTCTTCTACCTCTAGCAAA | 18 | |
| Reverse | AG | | |
| Fand2, | | | |
| Forward | TAATGGCCTGGAGTCCTACAC | 19 | Walter, D. et al., Nature, 2015 |
| Fand2, | CTCTTGGAGTAAAATGTGCCC | 20 | |
| Reverse | A | | |
| Xrcc5, | GACTTGCGGCAATACATGTTT | 21 | Flach, J. et al., Nature, 2014 |
| Forward | TC | | |
| Xrcc5, | AAGCTCATGGAATCAATCAGA | 22 | |
| Reverse | TCA | | |
| Atm, | TTAAGCATTCCTCCCAAGCA | 23 | Inoue, S. et al., Cancer Cell, 2016 |
| Forward | | | |
| Atm, | GCAAGCATGGTTCTTTGTGA | 24 | |
| Reverse | | | |
| Il6, | AGTTGCCTTCTTGGGACTGA | 25 | Inoue, S. et al., Cancer Cell, 2016 |
| Forward | | | |
| Il6, | TCCACGATTTCCCAGAGAAC | 26 | |
| Reverse | | | |
| Tnf, | AGGGTCTGGGCCATAGAACT | 27 | https://mouseprimerdepot.nci.nih. |
| Forward | | | gov/ |
| Tnf, | CCACCACGCTCTTCTGTCTAC | 28 | |
| Reverse | | | |
| Ccl2, | ATTGGGATCATCTTGCTGGT | 29 | https://mouseprimerdepot.nci.nih. |
| Forward | | | gov/ |
| Ccl2, | CCTGCTGTTCACAGTTGCC | 30 | |
| Reverse | | | |
| Ccl4, | GAAACAGCAGGAAGTGGGAG | 31 | https://mouseprimerdepot.nci.nih. |
| Forward | | | gov/ |
| Ccl4, | CATGAAGCTCTGCGTGTCTG | 32 | |
| Reverse | | | |
| Tlr4, | TGTCATCAGGGACTTTGCTG | 33 | https://mouseprimerdepot.nci.nih. |
| Forward | | | gov/ |
| Tlr4, | TGTTCTTCTCCTGCCTGACA | 34 | |
| Reverse | | | |
| Ticam1, | TGTCCAGCGGTGTGTTACAT | 35 | https://mouseprimerdepot.nci.nih. |
| Forward | | | gov/ |
| Ticam1, | CAGCCACCTAGAGATCAGCC | 36 | |
| Reverse | | | |
| Nfkb1, | GAACGATAACCTTTGCAGGC | 37 | https://mouseprimerdepot.nci.nih. |
| Forward | | | gov/ |
| Nfkb1 | CATCACACGGAGGGCTTC | 38 | |
| Reverse | | | |
| Nfkbiz | TATCGGGTGACACAGTTGGA | 39 | https://mouseprimerdepot.nci.nih. |
| Forward | | | gov/ |
| Nfkbiz, | TGAATGGACTTCCCCTTCAG | 40 | |
| Reverse | | | |
| Apex1, | AGCACTTGGTCTCTTGGAGG | 41 | https://mouseprimerdepot.nci.nih. |
| Forward | | | gov/ |
| Apex1, | GCAAATCTGCCACACTCAAG | 42 | |
| Reverse | | | |
| Stat1, | CTGAATATTTCCCTCCTGGG | 43 | https://mouseprimerdepot.nci.nih. |
| Forward | | | gov/ |
| Stat1, | TCCCGTACAGATGTCCATGAT | 44 | |
| Reverse | | | |
| Stat3, | CTGCTCCAGGTAGCGTGTGT | 45 | https://mouseprimerdepot.nci.nih. |
| Forward | | | gov/ |
| Stat3, | CTCAGCCCCGGAGACAGT | 46 | |
| Reverse | | | |
| Ly6a, | GGCAGATGGGTAAGCAAAGA | 47 | https://mouseprimerdepot.nci.nih. |
| Forward | | | gov/ |
| Ly6a, | CAATTACCTGCCCCTACCCT | 48 | |
| Reverse | | | |

Expression of β-Actin was used as an internal control using: Forward primer, 5′-GACGGCCAGGTCATCACTATTG-3′ (SEQ ID NO: 49) and Reverse primer, 5′-AGGAAGGCTGGAAAAGAGCC-3′ (SEQ ID NO: 50).

Staining with an Annexin-V and 7-AAD kit (BioLegend, Cat #640922) was performed according to the manufacturer's instruction for apoptosis analysis, along with labeling of LSK cells. For intracellular flow cytometry (IFC), BM cells were pre-stained by indicated cell-surface markers and then fixed by BD Cytofix/Cytoperm™ Kit (BD, Cat. No. 554714) (fixation and wash were performed according to the manufacturer's instruction) prior to being stained by antibodies of Ki-67 or γH2AX (cells were stained by these two markers for overnight) or by antibodies of cytokines (IL-6, TNFα, IL-1β or GM-CSF; cells were stained by these cytokine markers for 30 minutes). After intracellular staining, cells were washed three times by Cytoperm/wash buffer before being analyzed by flow cytometry.

Multiplex cytokine assays. Serum samples were prepared from PB (tail-bleeding) and diluted in sterile PBS (1 to 2 dilution(s)). Thirty-one cytokines or chemokines were quantified by multiplex immunoassay with a BioPlex 200 instrument (Eve Technologies, Mouse Cytokine Array/Chemokine Array 31-Plex, Cat # MD31).

Isolation of Lin-negative BM cells, LSK cells and qRT-PCR assays. Lin-negative BM cells (˜1×10⁶) were purified by an EasySep™ Mouse Hematopoietic Progenitor Cell Isolation Kit (StemCell, Cat #19856) according to the manufacturer's instruction. LSK cells were purified from Lin-negative BM cells by staining the cells with antibodies against c-Kit and Sca-1 followed by sorting them (Fluorescence-activated cell sorting (FACS) (BD FACSARIA)). Total RNA was extracted from Lin-negative cells by an RNeasy Mini Kit (Qiagen, Cat #74104) according to the manufacturer's instruction. Isolated RNA was quantified by spectrophotometry and RNA concentrations were normalized. cDNA was synthesized by SuperScript II Reverse Transcriptase (ThermoFisher Scientific, Cat #18064014). Resulting cDNA was analyzed by SYBR Green master mix (Life Technologies, Cat #4385612) with indicated primers on a ViiA7 Real-Time PCR instrument. Expression of β-Actin was used as internal control (Forward, 5′-GACGGCCAGGTCATCACTATTG-3′ (SEQ ID NO:49) and Reverse, 5′-AGGAAGGCTGGAAAAGAGCC-3′ (SEQ ID NO:50)) for calculating fold changes of indicated genes. A full list of qRT-PCR primers is provided in Table 2.

TABLE 2
Flowcytometry antibodies

| | Vendor | Cat # |
|---|---|---|
| Linage labeling | | |
| TER-119, PE | BioLegend | 116208 |
| Gr1, PE | BioLegend | 108408 |
| Mac1, PE | BioLegend | 101208 |
| B220, PE | BioLegend | 103208 |
| CD3 | BioLegend | 100206 |
| CD4 | BioLegend | 116006 |
| CD8a, PE | BioLegend | 100708 |
| LSK/HSC labeling | | |
| Lin, PE | BioLegend | cocktail as shown above |
| c-Kit, APC | BioLegend | 105812 |
| Sca-1, APC/Cy7 | BioLegend | 108126 |
| CD150, PE/Cy5 | BioLegend | 115912 |
| CD48, PE/Cy7 | BioLegend | 103424 |
| CMP/GMP/MEP/CLP | | |
| labeling | | |
| Lin, PE | BioLegend | cocktail as shown above |
| c-Kit, APC | BioLegend | 105812 |
| Sca-1, APC/Cy7 | BioLegend | 108126 |
| CD127, PE/Cy5 | BioLegend | 135016 |
| CD16/32, PE/Cy7 | BioLegend | 101318 |
| CD34, FITC | eBioscience | 11-03431-85 |
| Apoptosis labeling | | |
| Lin, PE | BioLegend | cocktail as shown above |
| c-Kit, APC | BioLegend | 105812 |
| Sca-1, APC/Cy7 | BioLegend | 108126 |
| Annexin V, FITC | BioLegend | 640906 |
| 7-AAD | BioLegend | 79993 |
| Intracellular Flow cytometry | | |
| (IFC) | | |

Cells were pre-stained by the cocktails for LSK/HSC labeling
Then fixed by BD CytoFix/CytoPerm buffer and washed by
CytoPerm/Wash Buffer
Then stained by indicated markers for proper duration

| | | |
|---|---|---|
| IL-6, AF488 | BD | 561363 |
| TNFa, AF488 | BioLegend | 506315 |
| IL-1b, FITC | eBioscience | 11-7114-80 |
| GM-CSF, FITC | BioLegend | 505403 |
| Ki67, FITC | BioLegend | 652410 |
| γH2AX, AF488 | BioLegend | 613406 |
| CD45.2/CD45.1 Chimerism | | |
| analysis | | |
| Overall chimerism analysis | | |
| CD45.2, PerCP/Cy5.5 | BioLegend | 109928 |
| CD45.1, PE/Cy7 | BioLegend | 110730 |
| Chimerism in hspcs | | |
| Lin, PE | BioLegend | Cocktail as shown above |
| c-Kit, APC | BioLegend | 105812 |
| Sca-1, APC/Cy7 | BioLegend | 108126 |
| CD45.2, PerCP/Cy5.5 | BioLegend | 109928 |
| CD45.1, PE/Cy7 | BioLegend | 110730 |
| Chimerism in mature cells | | |
| Mac1, PE | BioLegend | 101208 |
| B220, APC | BioLegend | 115512 |
| CD3, FITC | BioLegend | 100204 |
| CD45.2, PerCP/Cy5.5 | BioLegend | 109928 |
| CD45.1, PE/Cy7 | BioLegend | 110730 |
| Analysis of linage | | |
| development | | |
| Erythroid cell development | | |
| TER-119, PE | BioLegend | 116208 |
| CD71, APC | eBioscience | 17-0711-82 |
| Myeloid, B-cell, T-cell | | |
| development | | |
| Mac1, PE | BioLegend | 101208 |
| B220, APC | BioLegend | 115512 |
| CD3, FITC | BioLegend | 100204 |

CFU assay. Bone marrow Lin-negative cells or LSK cells were isolated as described above and platted in a CFU assay using MethoCult™ GF M3434 (Stem Cell). Colonies were counted after 7-days of culture.

CHIP-qPCR assay. BM Lin-negative cells were used to extract chromatin DNA using MAGnify™ Chromatin Immunoprecipitation System (ThermoFisher) according to the manufacturer's instruction. CHIP purified chromatin DNA and input DNA were normalized to identical concentration for qPCR validation and enrichment analysis (1% enrichment of input level was defined as unit 1). The following antibodies were used for chromatin precipitation:Anti-IκBζ and Anti-Stat3 (Cell Signaling Technologies). Primers for CHIP-qPCR analysis are listed in Table 3.

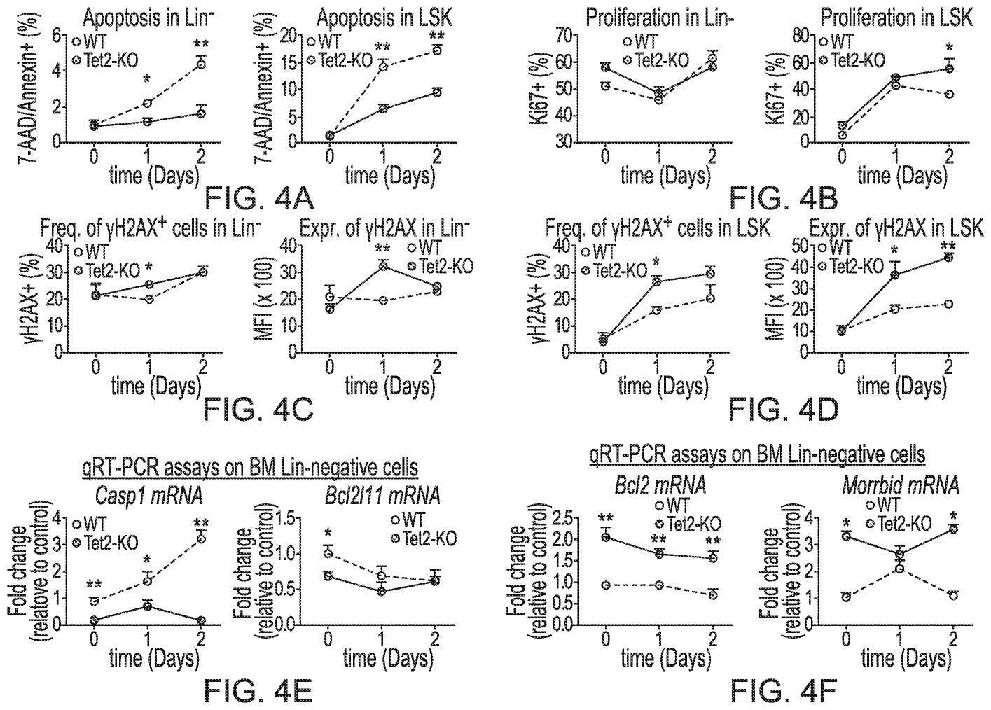

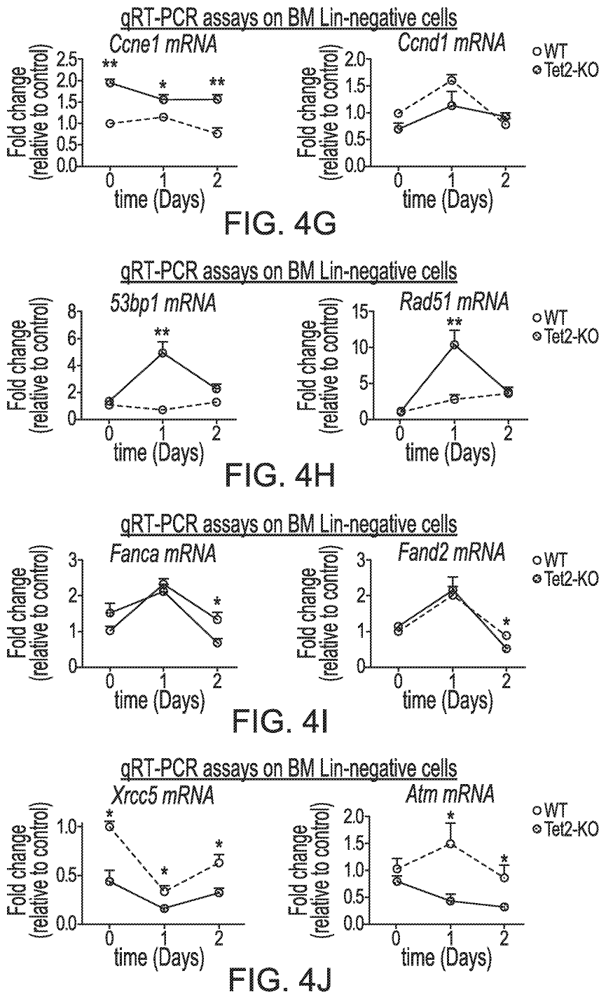

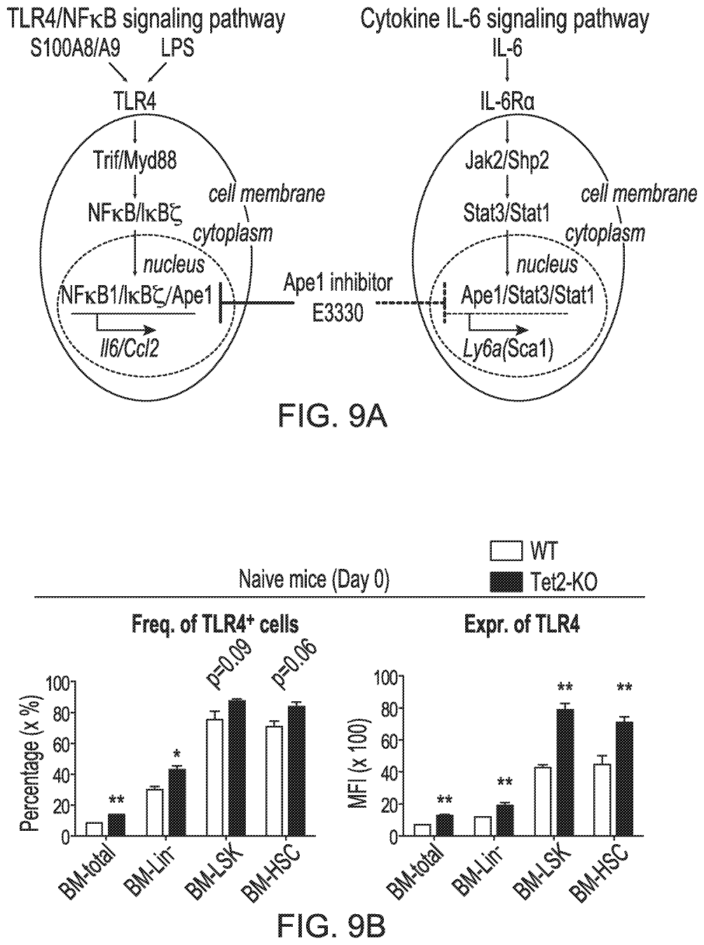

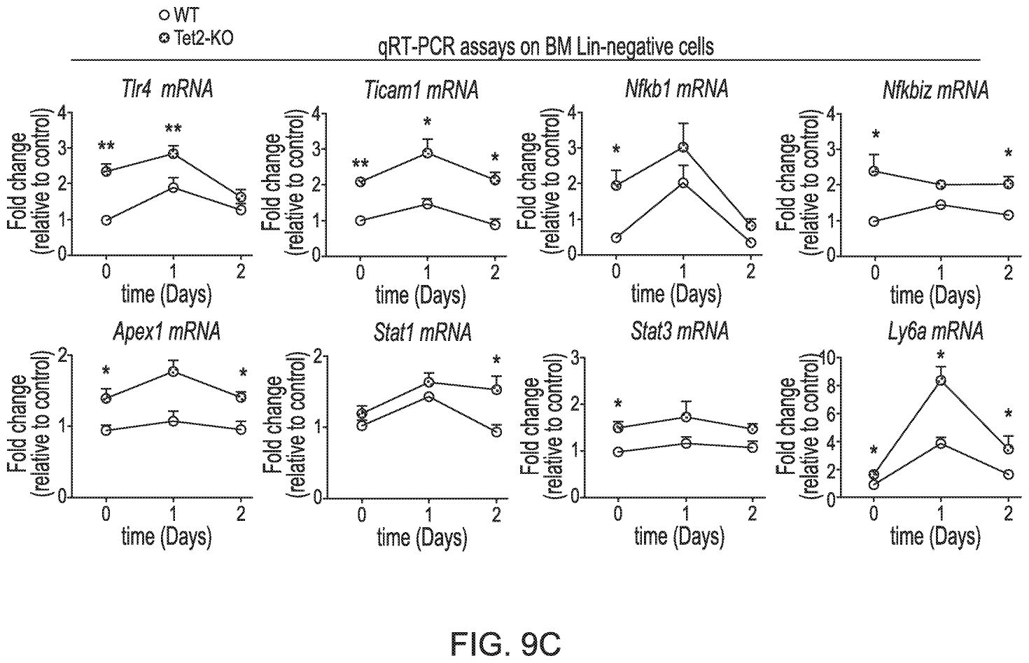

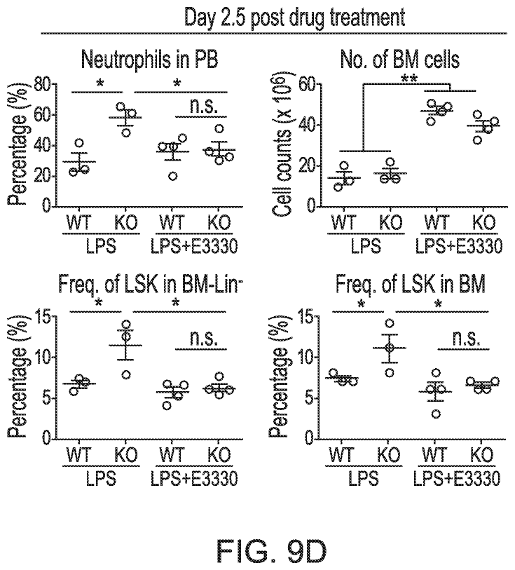

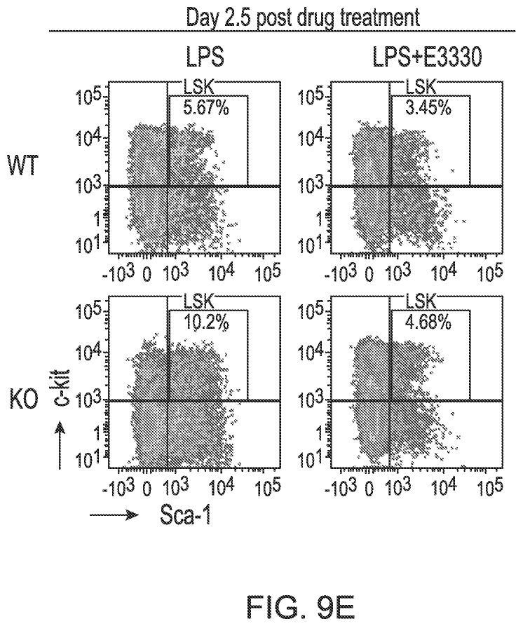

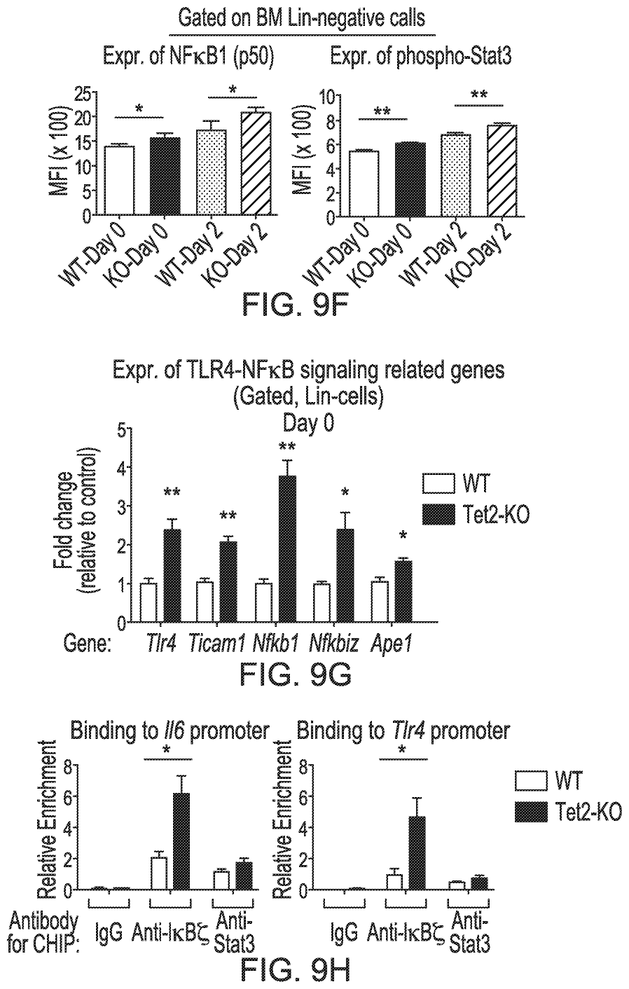

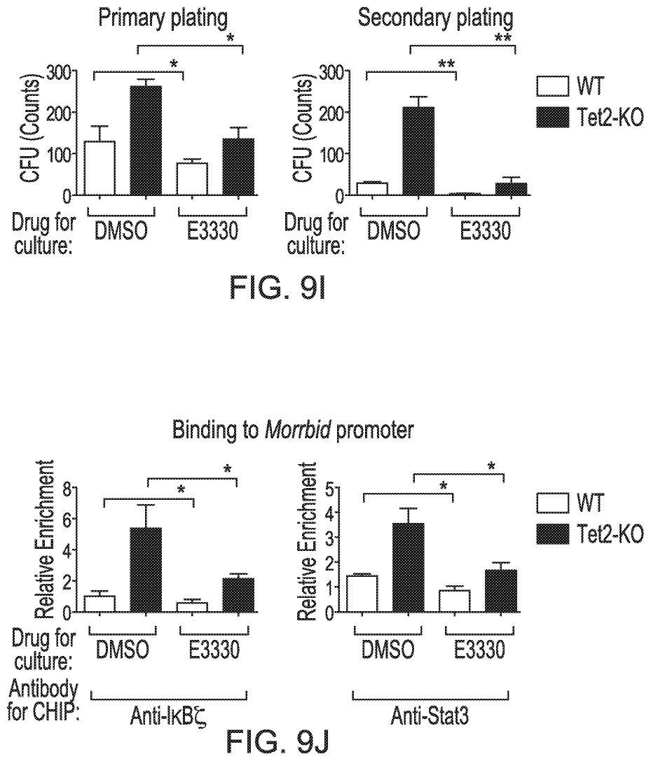

TABLE 3
CHIP-qPCR primers used in FIGS. 4 and 9

| qRT-PCT primers | | |
|---|---|---|
| used in FIGS. 4 & 9 | SEQ ID NO | Source |
| Promoter of IL-6, | CCTGCGTTTAAATAACATCAGCTTTAGCTT | Zhang, Q. et al., Nature 2015 |
| Forward | (SEQ ID NO: 51) | |
| Promoter of IL-6, | GCACAATGTGACGTCGTTTAGCATCGAA | |
| Reverse | (SEQ ID NO: 52) | |
| Promoter of TLR4, | CACAAGACACGGCAACTGAT | Pedchenko, T.V. et al., AJP Lung, |
| Forward | (SEQ ID NO: 53) | 2005 |
| Promoter of TLR4, | TCGCAGGAGGGAAGTTAGAA | |
| Reverse | (SEQ ID NO: 54) | |
| Promoter of Morrbid, | AGCACGAGTCATCTGGTTCC | Kotzin, J. J. et al., Nature, 2016 |
| Forward | (SEQ ID NO: 55) | |
| Promoter of Morrbid, | ACCCAGTCCCCTCATTCTCA | |
| Reverse | (SEQ ID NO: 56) | |

Competitive bone marrow transplantation (cBMT). B6.SJL-Ptprc^{a} Pepc^{b}/Boy (BoyJ, CD45.1) were purchased from The Jackson Laboratory. Recipient animals (F1, CD45.2/CD45.1) were generated by crossing C57BL/6 (CD45.2) with BoyJ (CD45.1). For primary cBMT, CD45.2 donor BM cells from naïve or LPS-treated mice were mixed equally with BoyJ CD45.1 competitor BM donor cells (with an equal number of viable total cells, 500K:500K) prior to intravenous (i.v.) tail injection into lethally irradiated F1 CD45.2/CD45.1 recipient (700 cGy plus 400 cGy). For secondary cBMT, donor BM cells from primary cBMT recipients were mixed with BoyJ CD45.1 competitor BM cells (with equal number of viable total cells) prior to intravenous (i.v.) tail injection into lethally irradiated F1 CD45.2/CD45.1 recipient (700 cGy plus 400 cGy). For LSK cell engraftment, 2000 LSK cells from LPS treated or control mice were mixed with 500,000 (2K:500K) BoyJ CD45.1 supporting cells and injected into F1 mice as described above. Chimerism analysis for progressive engraftment was run on PB samples monthly (every 4-week interval) post BM transplantation. End-point chimerism analysis was based on various fractions of BM cells from the recipients.

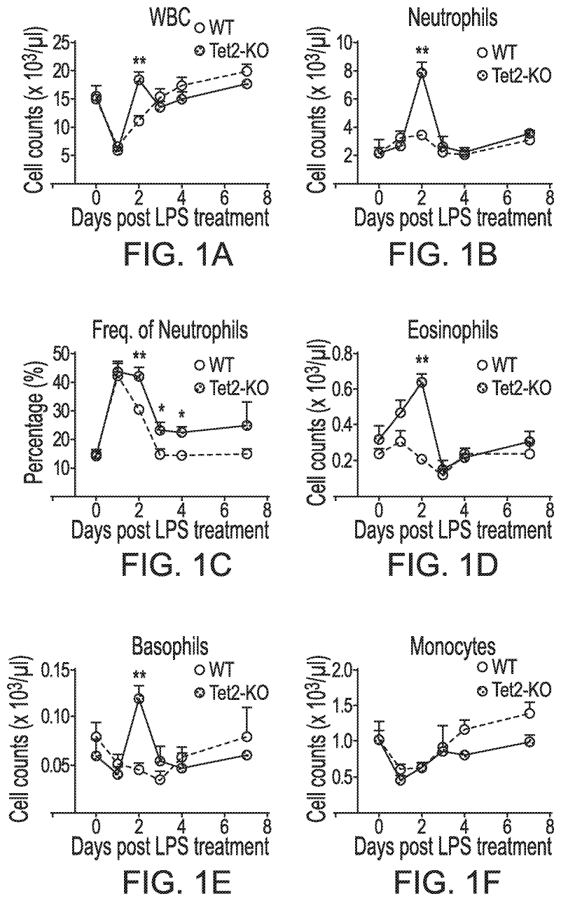

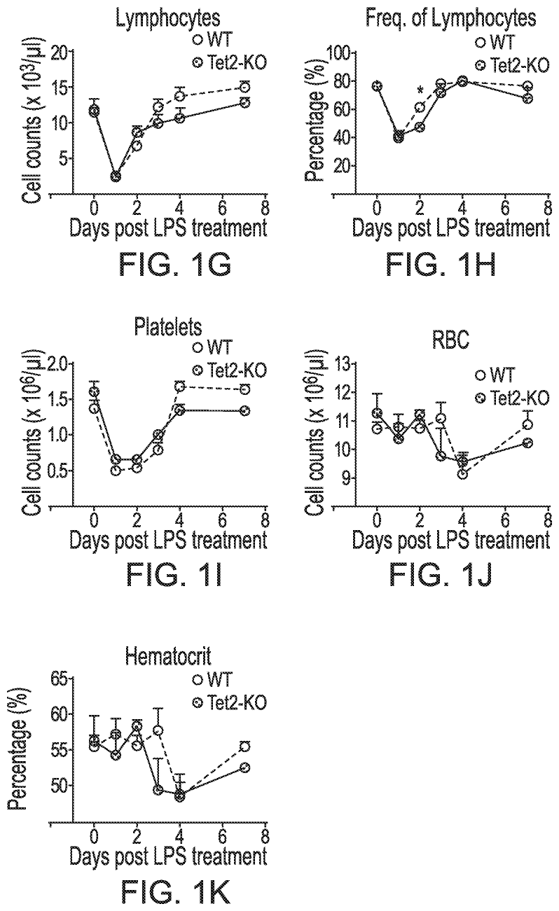

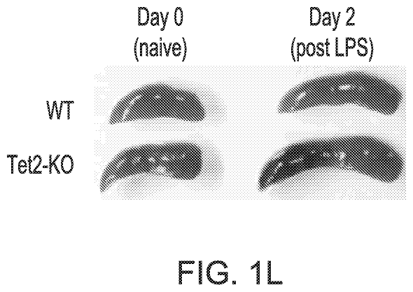

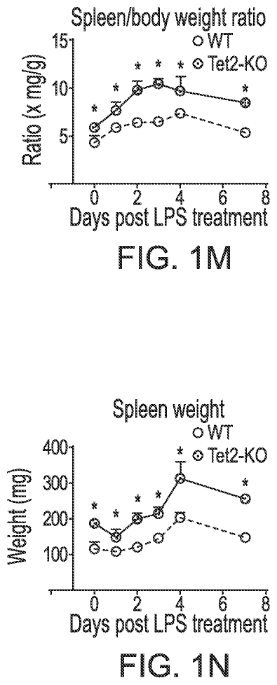

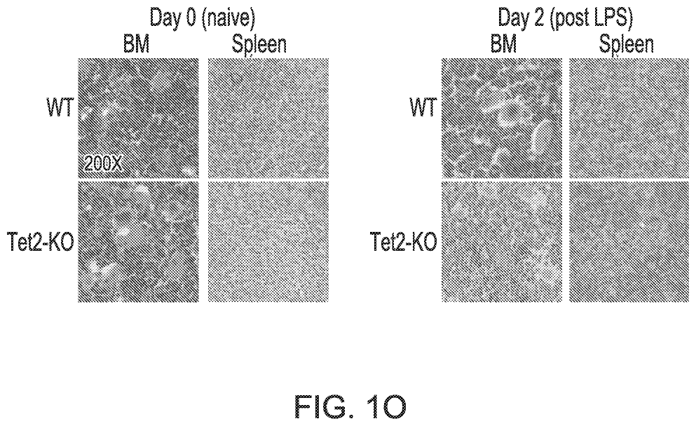

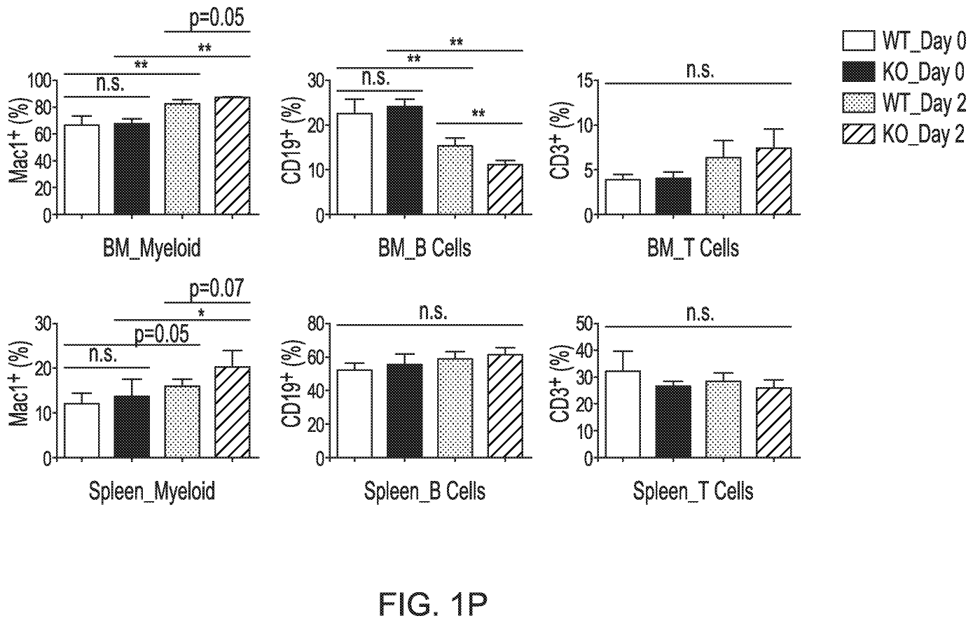

Statistics. All experimental procedures on Tet2-KO samples were run in parallel with wildtype controls (sex and age matched littermate controls when possible) for observing experimental variabilities. Analysis of grouped data was not blinded and no samples were excluded. Aged Tet2-KO mice were randomized into two groups for treatment with APX3330 or vehicle, SHP099 or vehicle (FIGS. 11A-11D). P-value was calculated using an unpaired t-test for comparing means of two groups (GraphPad Prism 6.0). Error bars in FIGS. 1-10 and 11A indicate the standard error of mean (s.e.m.) while error bars in FIG. 11C indicate the standard deviation (s.d.). Except that the secondary cBMT was performed in only one independent experiment for each group, all other assays were performed in at least two independent experiments with at least 3-5 biological replicates in total.

### Results

Rapid and Extended Granulopoiesis in TET2-KO Mice in Response to an Acute Inflammatory (LPS) Challenge

Consistent with previous studies, developmental defects, including hematopoiesis, were not observed in the naïve TET2-KO mice by flow cytometry analysis or by blood count for hematologic parameters at the age of 2-3 months old compared with wild type. Lipopolysaccharide (LPS), a ligand that functions by stimulating Toll-like receptor 4 (TLR4)/NFκB signaling, is wildly used as an efficient chemical drug for inducing acute inflammation in mice. To test whether Tet2-deficient preleukemic stem, progenitor and mature cells respond to acute inflammation, LPS was injected into Tet2-KO mice and their wildtype counterparts and these mice were followed for 7 days to assess changes in peripheral blood (PB) hematologic parameters. At Day 2 (48 hours after LPS treatment), it was observed in Tet2-KO, a significantly rapid and enhanced recovery of white blood cells (WBC), which is most attributed to an increase in the absolute number as well as in the percentage of neutrophils (NE) (FIG. 1A-1C). The difference between wild type and TET2-KO in response to acute inflammation becomes more dramatic when the percentages of NE in WBC were examined at Day 2 to Day 4 after LPS treatment (FIG. 1C). The increase in neutrophil percentage in Tet2-KO mice persisted for the entire follow up duration of the Example, demonstrating an extended “emergency granulopoiesis” in LPS treated Tet2-KO mice compared to controls. The cell numbers of eosinophils (EO) and basophils (BA), but not monocytes (MO), were elevated in TET2-KO at Day 2 after LPS treatment (FIGS. 1D-1F). The cell numbers of lymphocytes (LY), platelets (PL) and red blood cells (RBC) were altered in a similar pattern in wild type and TET2-KO with no significant differences, while lymphocyte percentage in Tet2-KO mice was significantly reduced compared to controls on Day 2 post LPS challenge (FIGS. 1G-1K). Furthermore, a significant increase in spleen weight was also observed in Tet2-KO mice relative to controls at every time point examined (FIGS. 1L-1N). Although no gross changes in the bone marrow and splenic pathology were noted post LPS treatment, the frequency of myeloid mature cells (Mac1+) was increased in Tet2-KO mice compared to controls (FIGS. 1O and 1P). Taken together, these data suggest that Tet2-KO mice manifest an amplified and sustained “emergency granulopoiesis” in response to an acute inflammatory challenge.

Acute Inflammatory Challenge Results in Enhanced Numbers of Myeloid Progenitors and Hematopoietic Stem Cells in Tet2-KO Mice

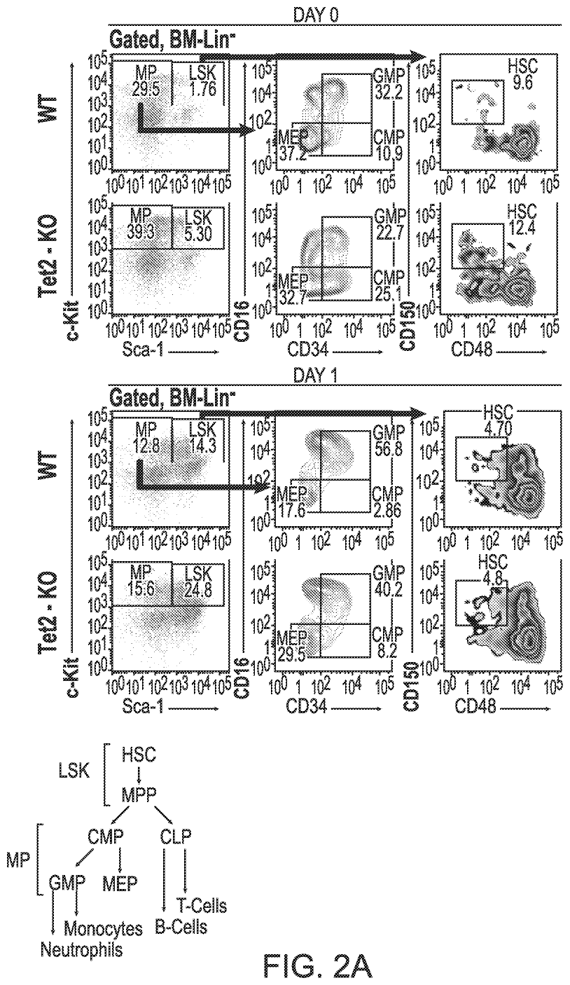

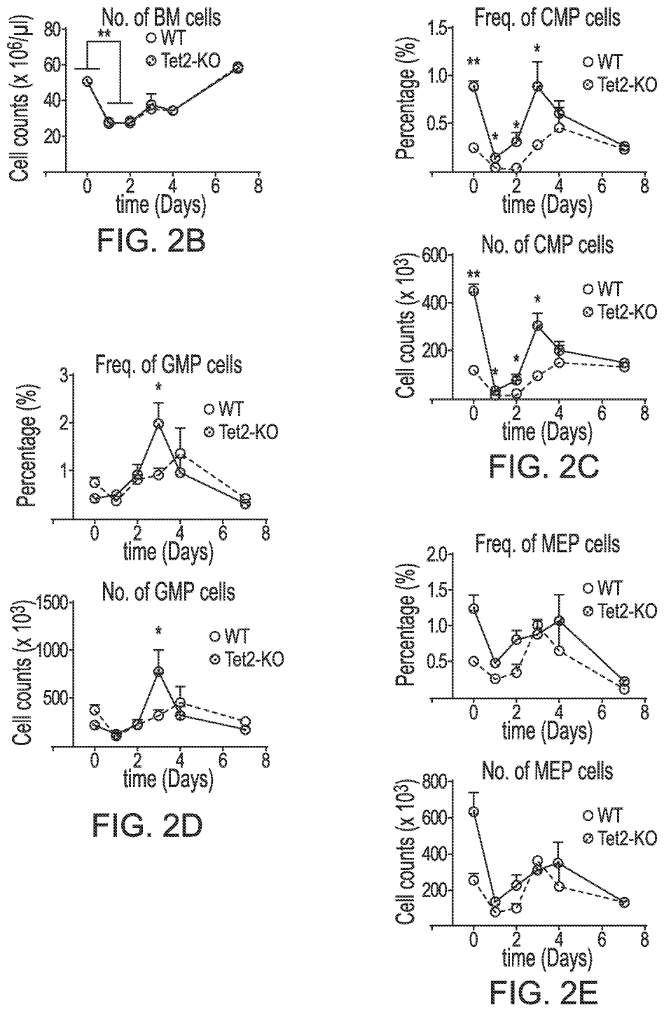

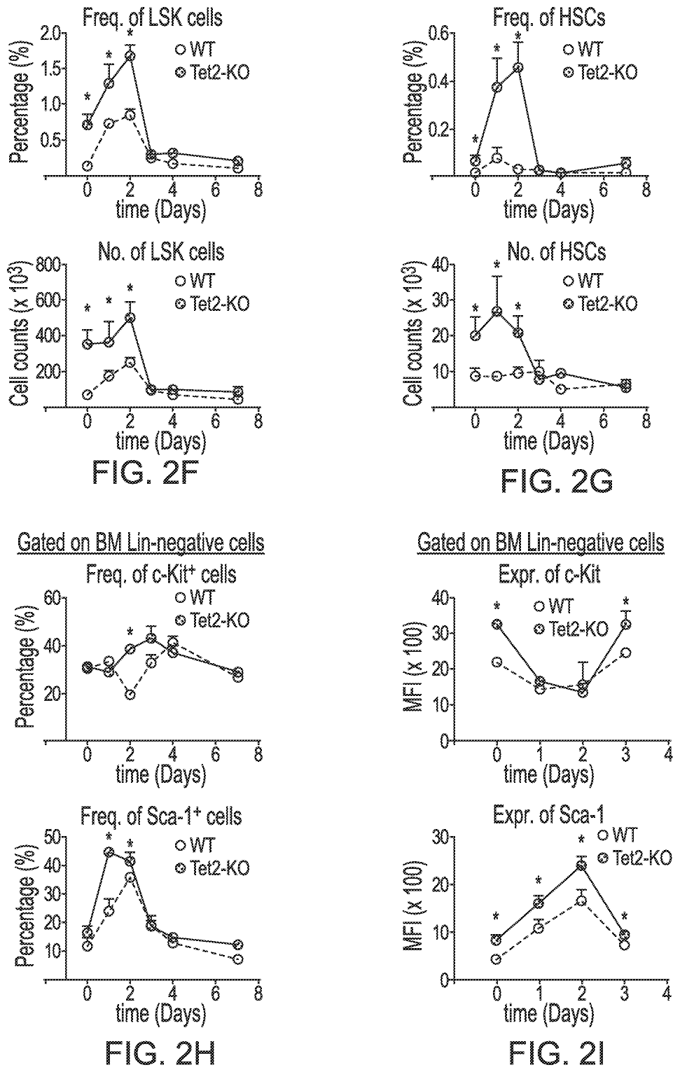

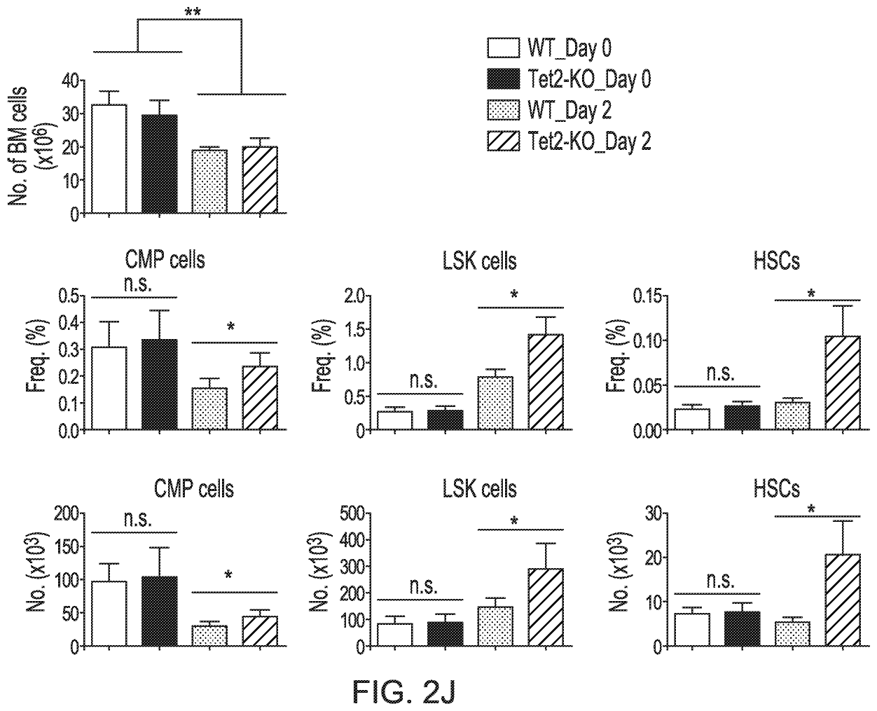

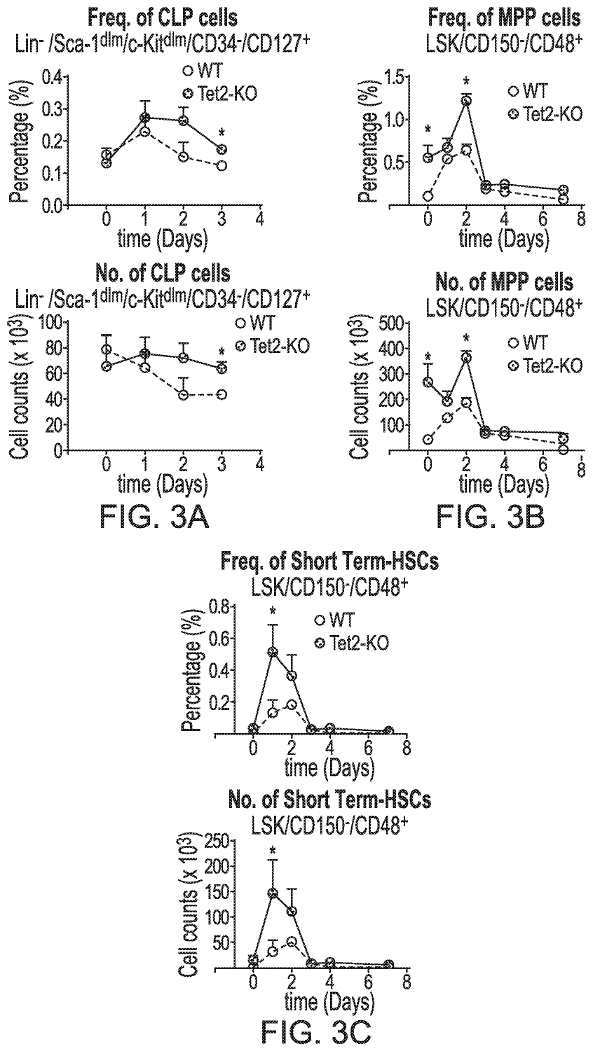

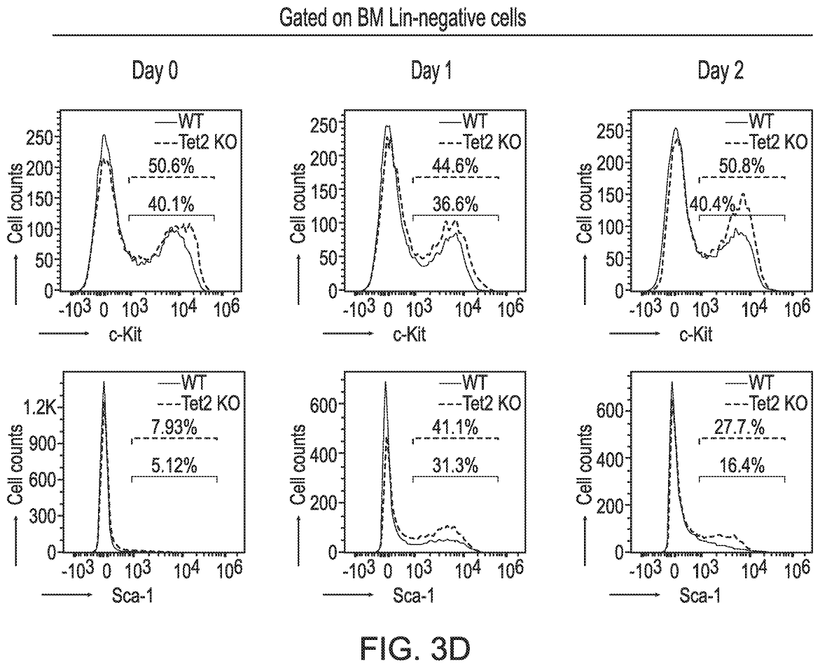

Infection induces acute inflammation and activates hematopoiesis at the levels of both hematopoietic stem cells (HSC) and progenitor cells (HPC) to adapt to the pathological insure. In contrast to steady state hematopoiesis (naïve, non-infection), infection-induced hematopoiesis in the bone marrow is recognized as “emergency hematopoiesis”. Post LPS challenge, the LSK compartment and HSC compartment (LSK/CD48⁻/CD150⁺), in addition to various progenitor compartments containing common myeloid progenitors (CMP, Lit⁻/Sca-1⁻/cKit⁺/CD16⁻/CD34⁺), common lymphocyte progenitors (CLP, Lin⁻/Sca-1^{dim}/c-Kit^{dim}/CD127⁺/CD34⁻), granulocyte-macrophage progenitor (GMP, Lin⁻/Sca-1⁻/cKit⁺/CD16⁺/CD34⁺), megakaryocyte-erythroid progenitor (MEP, Lin⁻/Sca-1⁻/c-Kit⁺/CD16⁻/CD34⁻) were analyzed in bone marrow by flow cytometry (gating strategies shown in FIG. 2A). A similar drop in the overall bone marrow cellularity in both wildtype (WT) and Tet2-KO mice post LPS challenge was observed (FIG. 2B). In contrast to a lack of difference in the overall bone marrow cellularity between wildtype and the Tet2 mutant in response to LPS challenge, LPS induced acute inflammation resulted in significant differences in the recovery of various progenitors in the BM of Tet2-KO mice on Day 1 relative to controls (representative flow cytometry plots shown in FIG. 2A). Quantitatively, LPS challenge of Tet2-KO mice resulted in increased recovery of CMPs, GMPs and CLPs, but not MEPs, with regards to both frequency as well as absolute numbers relative to controls (FIGS. 2A, 2C-2E and FIG. 3A). The increase was more prominent in the GMP compartment (FIG. 2D). Post LPS treatment, a significant increase in the enrichment of LSKs and HSC frequency and numbers in the BM were also observed in Tet2-KO mice compared to controls (FIGS. 2A, 2F and 2G) on Day 1 and Day 2 post LPS treatment. Similarly, Tet2-KO mice exhibited higher counts of multipotent progenitors (MPPs, defined by LSK/CD48⁺/CD150⁻) or short-term HSC (defined by LSK/CD48⁺/CD150⁺) during the early stages of the LPS challenge (FIGS. 3B & 3C). Given that adult (12-16 week), Tet2-KO mice manifest increased basal (Day 0, before LPS challenge) levels of CMPs, LSKs and HSCs compared to controls, it was assessed whether younger juvenile Tet2-KO mice, which show similar frequency and numbers of CMPs, LSKs and HSCs as wildtype, responded to LPS challenge in a manner similar to older Tet2-KO mice. As shown in FIG. 2J, juvenile Tet2-KO mice also exhibit enhanced response to LPS challenge in all the bone marrow progenitor subsets examined including CMPs, LSKs and HSCs compared to controls. When examining day-to-day changes after LPS treatment, elevated Sca-1⁺, but not c-Kit⁺, hematopoietic progenitor cells within the lineage negative fraction (Lin-negative or Lin⁻) were observed in the bone marrow of both wildtype and Tet2-KO mice between Day 1 and Day 3 compared to Day 0 (FIGS. 2A, 2H and FIG. 3D). By assessing the expression of Sca-1 and c-Kit by mean fluorescence intensity (MFI) using flow cytometry, enhanced Sca-1 expression was consistently observed at all-time points from Day 0 to Day 3 in Tet2-KO mice compared to wildtype controls (FIG. 2I). Collectively, these data suggest that deficiency of Tet2 results in a higher capacity to turn on emergency hematopoiesis (activation or recovery of HSPCs and their differentiation into mature myeloid cells).

Hematopoietic Stem and Progenitor Cells Deficient in Tet2 Showed Increased Cell Survival, Proliferation and Enhanced DNA-Damage in Response to Acute Inflammation.

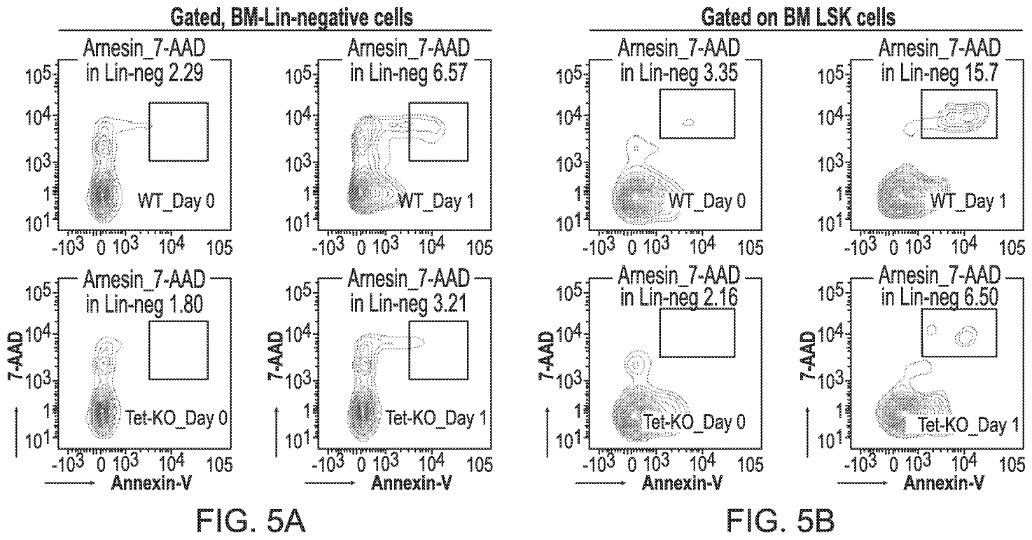

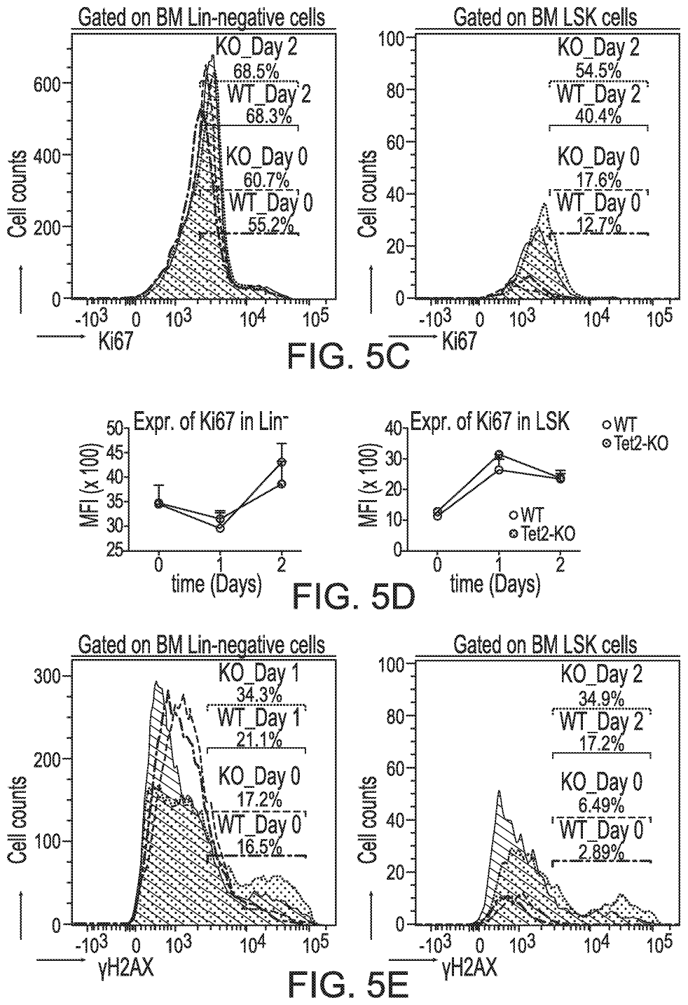

Given the observations of enhanced counts (activation or recovery) of HSPCs in the absence of Tet2 upon LPS challenge, it was determined if Tet2-deficient HSPCs responded differently to survival, growth and DNA-damage upon an inflammatory challenge. Apoptosis in HSPCs was examined by Annexin-V plus 7-AAD staining and flow cytometry (FIGS. 5A & 5B). The proliferation was determined in these cells by assessing the percentage of Ki67⁺ cells (intracellular staining and flow cytometry, IFC) (FIGS. 5C & 5D). During emergency hematopoiesis induced by LPS, Tet2-deficient hematopoietic progenitor cells maintained a lower level of apoptosis (defined by percentage of Annexin-V⁺/7-AAD⁺ cells) in both Lin-negative pool as well as in the LSK pool compared to wildtype controls (FIGS. 4A, 5A and 5B). Although no differences were observed in the percentage of cycling cells in the Lin-negative fraction of the bone marrow, the percentage of Ki67⁺ cells in the LSK pool was significantly higher in Tet2-KO mice compared to wildtype controls on Day 2 post LPS challenge (FIGS. 4B, 5C and 5D).

Recent studies have suggested that activation of HSCs from its dormancy may induce DNA damage under conditions of inflammation or stress. To assess if Tet2-deficient bone marrow cells are more susceptible to DNA damage upon LPS stimulation, the DNA damage response was analyzed in these cells by detecting histone H2A.X phosphorylation (γH2AX, a sensitive marker for DNA damage) through intracellular staining and flow cytometry. A higher expression was observed of γH2AX and a higher frequency of γH2AX⁺ cells in HSPC at early stages of emergency hematopoiesis (FIGS. 4C, 4D and 5E). The phenotypic observations with regards to enhanced cell survival, proliferation and DNA-damage were supported by changes in the expression of pro-apoptotic genes (Casp1, encoding Caspase 1, and Bcl2111, encoding Bim, exhibiting decreased expression in Tet2-deficient cells), pro-survival genes (Bcl2 and Morrbid, exhibiting increased expression in Tet2-deficient cells; Morrbid was a recently identified gene encoding a long-non-coding RNA, which specifically promotes the survival of myeloid cells including neutrophils), cell cycle genes (Ccne1, encoding CyclinE1, exhibiting increased expression in Tet2-deficient cells), DNA damage response genes (53bp1 and Rad51, exhibiting increased expression in Tet2-deficient cells) and genes encoding components for the DNA repair pathway (Fanca, Fancd2, Xrcc5 and Atm, exhibiting decreased expression in Tet2-deficient cells) (FIGS. 4E-4J). The expression of Ccnd1 (encoding CyclinD1), Cdkn1b (encoding cell cycle inhibitor p27) and Cdkn1c (encoding cell cycle inhibitor p57) were comparable between wildtype and Tet2-deficient cells during this time period (FIG. 4G and data not shown).

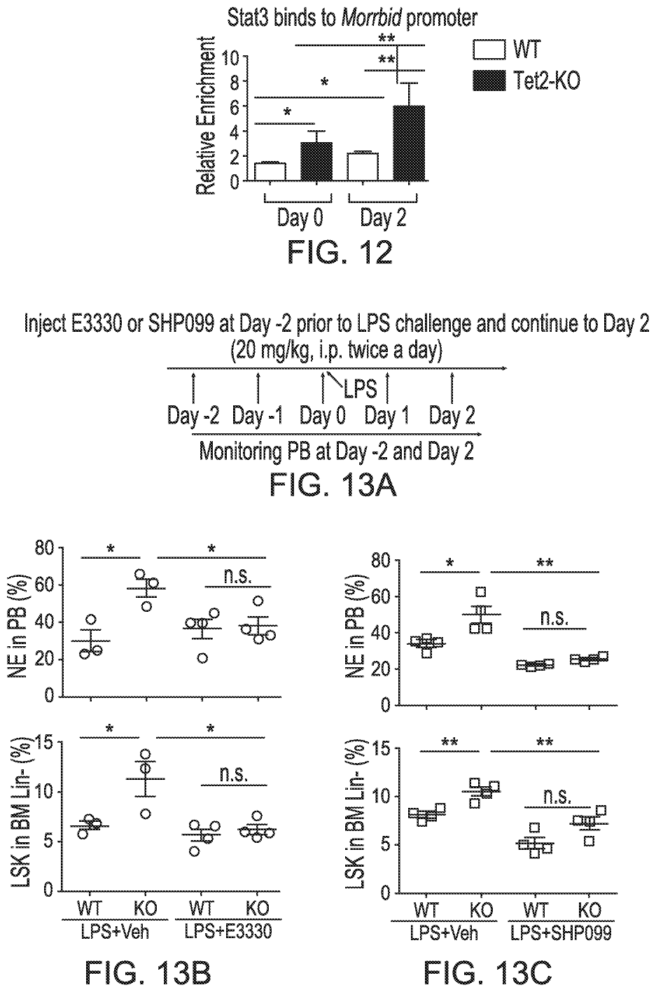

Given that the expression of cell apoptosis related genes was strikingly different between wild type and Tet2-KO cells, combined with the recent observation implicating a novel anti-apoptotic role of Morrbid in the regulation of myeloid cell survival, it was hypothesized that the expression of Morrbid is likely to be directly regulated by an essential inflammatory regulator such as Stat3. Stat3 activation is upregulated in naïve Tet2-KO HSPCs relative to controls (FIG. 9F). Furthermore, CHIP-qPCR analysis revealed a significant enrichment in the binding of Stat3 to the Morrbid promoter in Tet2-KO HSPCs compared to controls (FIG. 12). Taken together, these data suggest that Tet2-depleted HSPCs manifest a higher survival and proliferation rate, and may harbor increased DNA damage upon acute inflammatory challenge. Further, the enhanced survival of Tet2-KO HSPCs is likely due to enhanced and sustained upregulation of Morrbid via Stat3.

Differential Impact of Acute Inflammation on the Function of Normal Vs. Tet2-Deficient Hematopoietic Stem Cells.

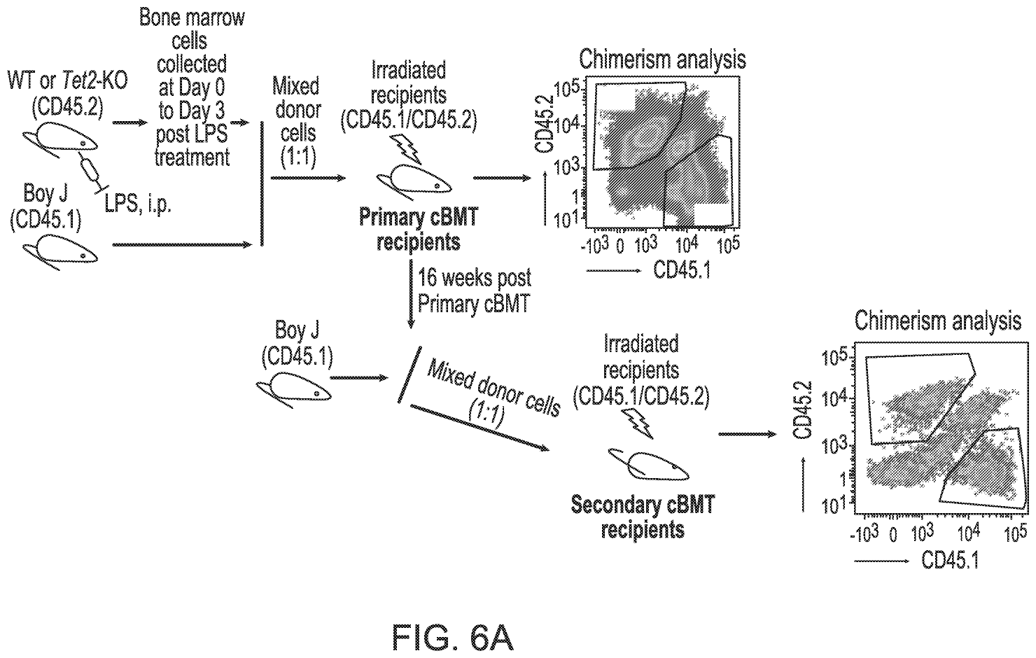

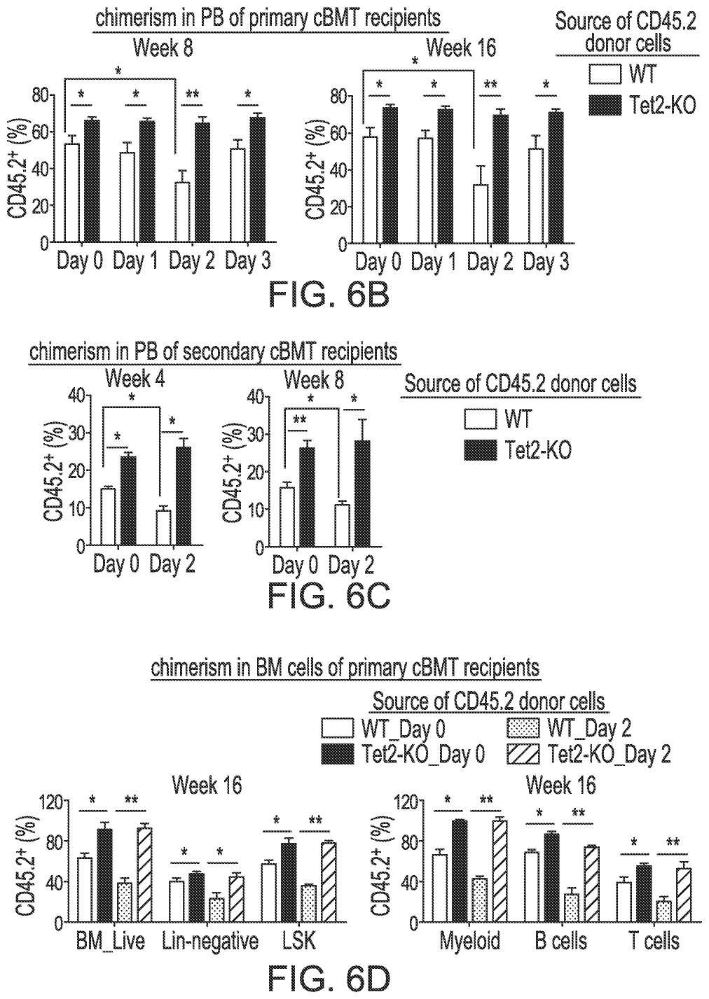

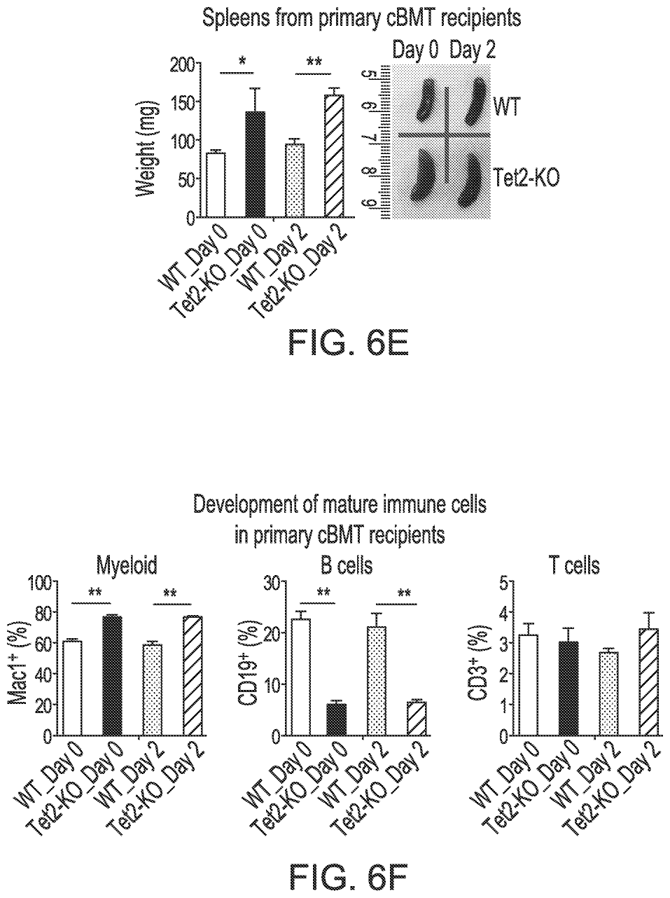

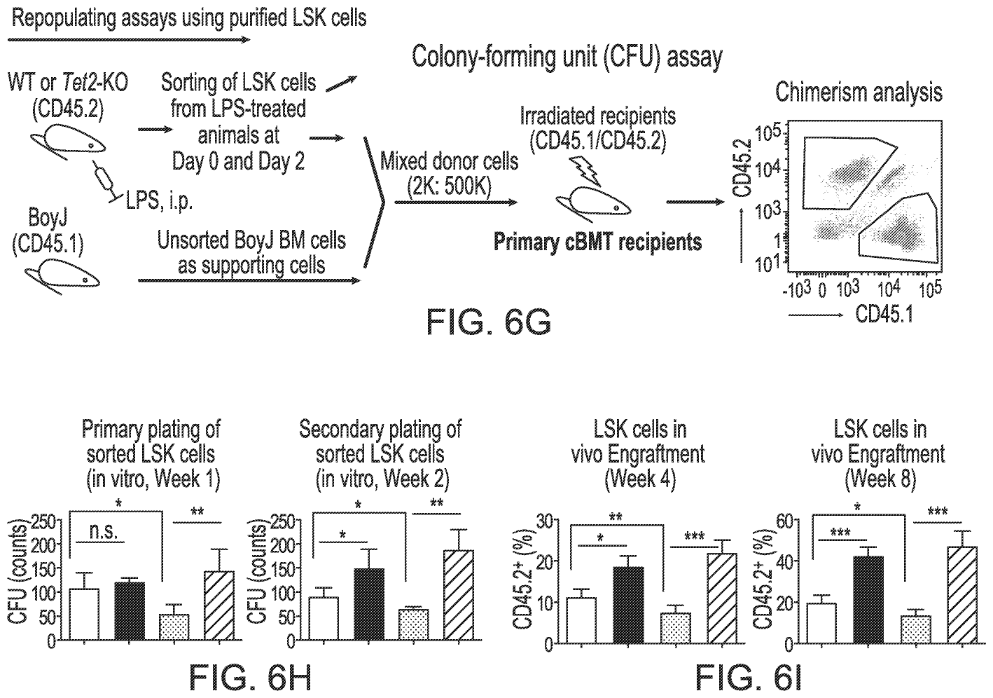

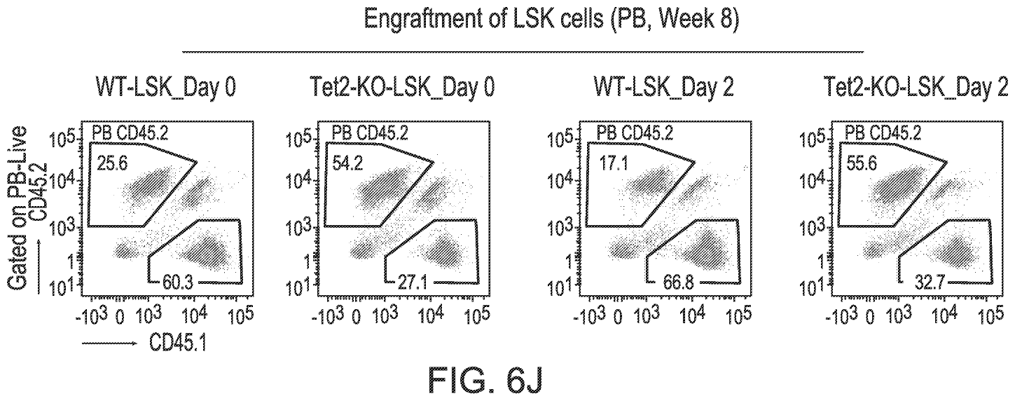

Recent studies have shown that LPS challenge or bacterial infection not only expands the HSC/LSK population, but also potentially depletes HSCs or impairs their self-renewing capability. It was analyzed whether LPS-challenged Tet2-deficient HSPCs would also demonstrate reduced stem cell activity after being exposed to inflammatory stress. To assess this, a competitive repopulation assay was performed using bone marrow viable total cells. The scheme for conducting competitive bone marrow transplantation (cBMT) is illustrated in FIG. 6A. Post cBMT chimerism analysis on CD45.2⁺ and CD45.1⁺ fractions of peripheral blood (PB) cells from primary recipients showed that the repopulating activity of CD45.2 donor cells from WT mice on Day 2 post LPS treatment (% CD45.2⁺ WT_Day 2 in primary recipients) was significantly reduced compared to naïve wildtype donor cells (% CD45.2⁺ WT_Day 0 in primary recipients) (% CD45.2⁺ WT_Day 0 vs. % CD45.2⁺ WT_Day 2, * P<0.05, FIG. 6B). In contrast, LPS-stressed Tet2-deficient CD45.2 donor cells did not show reduction in repopulating ability on any of the post LPS treatment time points examined (FIG. 6B). Further, at every time point examined, Tet2-deficient CD45.2 bone marrow donor cells demonstrated higher repopulating ability compared to WT CD45.2 donor controls, although the greatest difference was observed on CD45.2 chimerism primed with CD45.2 donor cells of Day 2 post LPS treatment (i.e., % CD45.2_WT_Day 2 vs. % CD45.2_Tet-KO_Day 2, ** P<0.01, FIG. 6B). Secondary cBMT experiments further confirmed that WT CD45.2 donor cells on Day 2 post LPS treatment showed decreased repopulating activity while Tet2-deficient CD45.2 donor cells were resistant to LPS stress and maintained robust repopulating and engraftment advantage (FIG. 6C).

Chimerism analysis of various fractions of BM populations including BM viable total cells (BM_Live), Lin-negative cells, LSK cells, myeloid cells (labeled by Mac1), B-cells (labeled by CD19) and T-cells (labeled by CD3) in the bone marrow of primary cBMT recipients also demonstrated that the repopulation of Tet2-deficient donor HSPCs was significantly higher than controls (*P<0.05, **P<0.01, FIG. 6C). Additionally, primary cBMT recipients transplanted with Tet2-KO cells exhibited splenomegaly, myeloid-lineage skewing and defective B-cell development in the bone marrow, indicating a cell-autonomous effect of Tet2-deficiency in leading to early pre-leukemic pathology (FIGS. 6D & 6E).

To further compare the repopulating activity of stem cells after LPS induced inflammatory damage in wildtype and Tet2-KO mice, identical numbers of LSK cells from pre- and post-LPS treated wildtype and Tet2-KO mice were sorted and subjected to colony forming unit assay (CFU assay) in vitro and bone marrow transplantation assay in vivo (FIG. 6G). Consistent with the data utilizing whole bone marrow cells (FIG. 6B), purified wildtype LSK cells derived from mice 2 days post LPS treatment, showed impaired colony forming ability in vitro and significantly reduced repopulating activity in vivo compared to Tet2-deficient LSK cells, which were significantly more resistant to inflammatory insult (FIGS. 6H-6J). Taken together, the cBMT and CFU assay functionally demonstrate that while the repopulating activity of wildtype HSPCs is significantly impaired in response to LPS-induced inflammatory stress, LPS-treated Tet2-deficient HSPCs maintain greater repopulation/engraftment and are resistant to inflammatory stress.

Tet2-KO Mice Show Enhanced Expression of Pro-Inflammatory Cytokines.

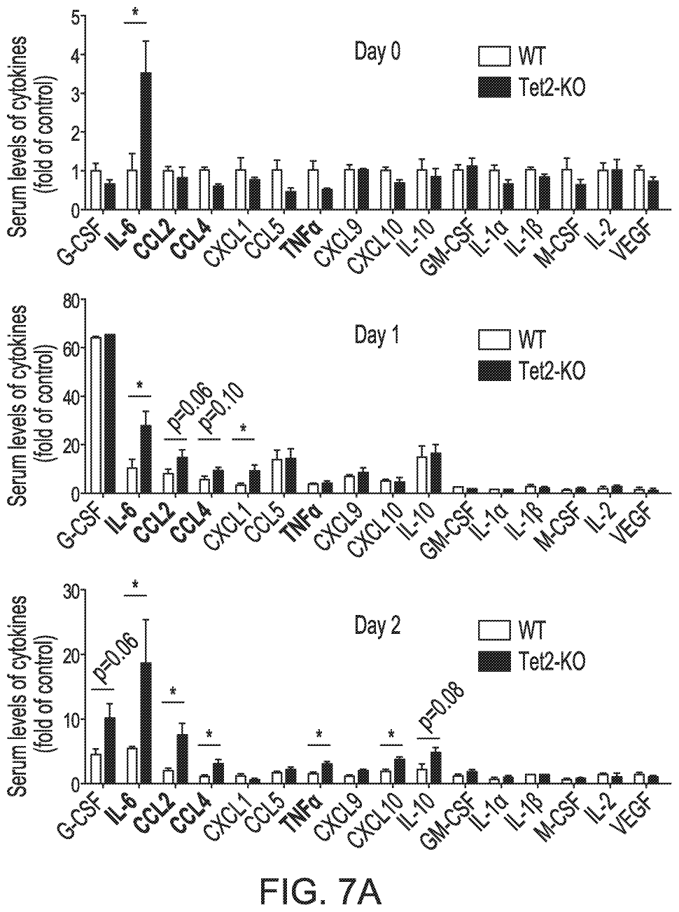

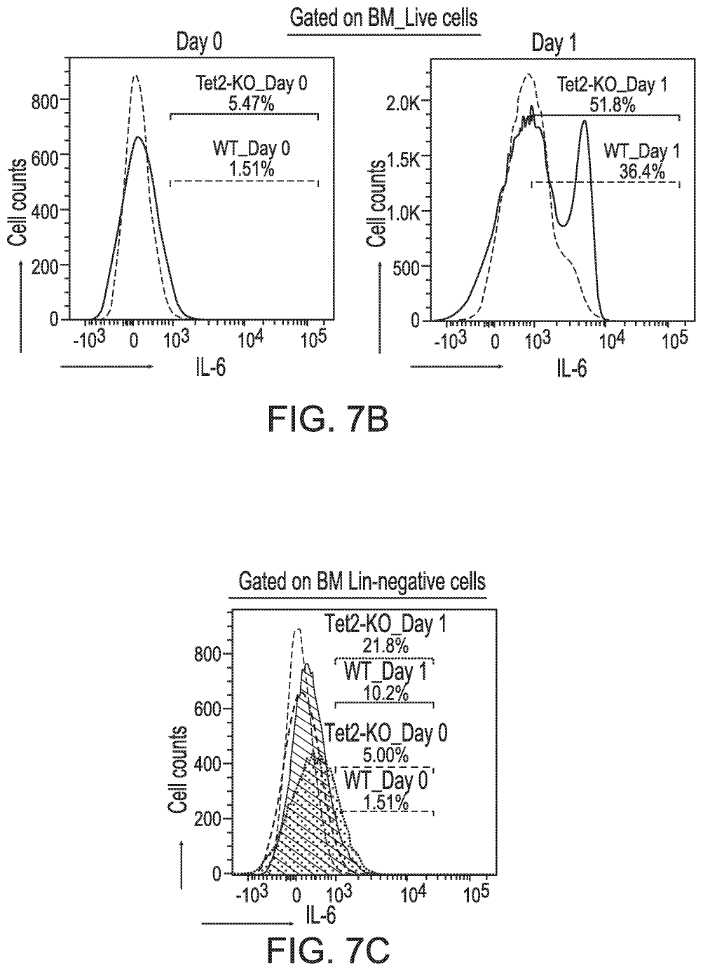

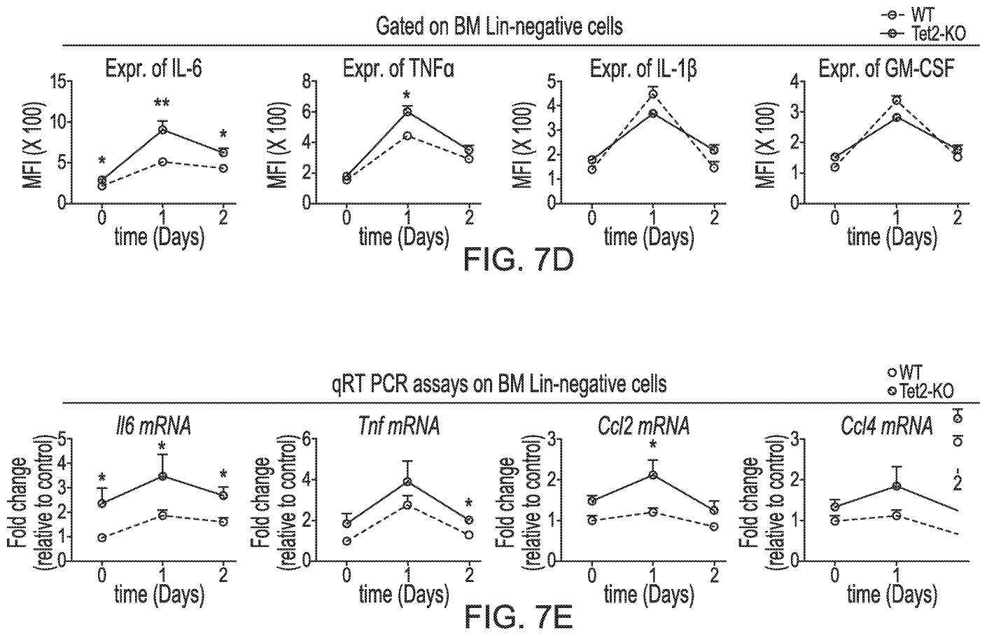

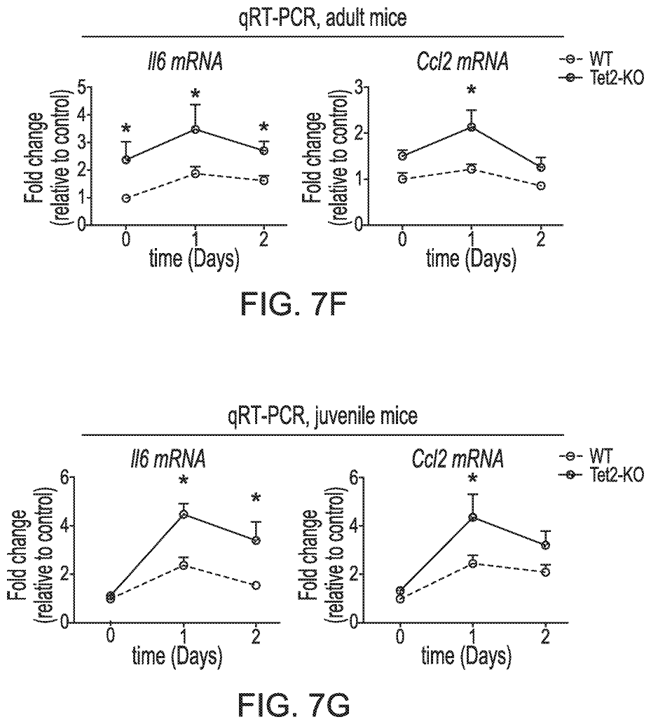

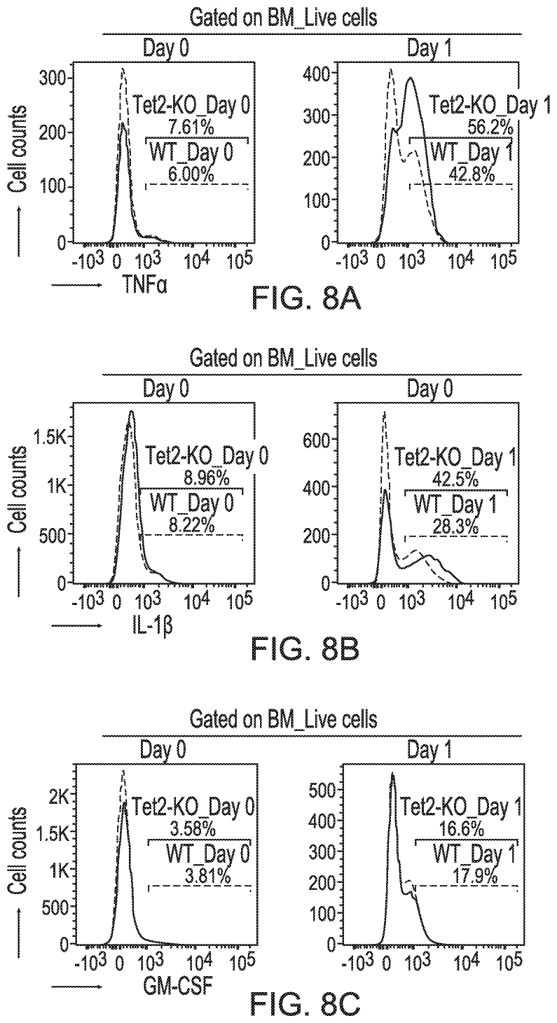

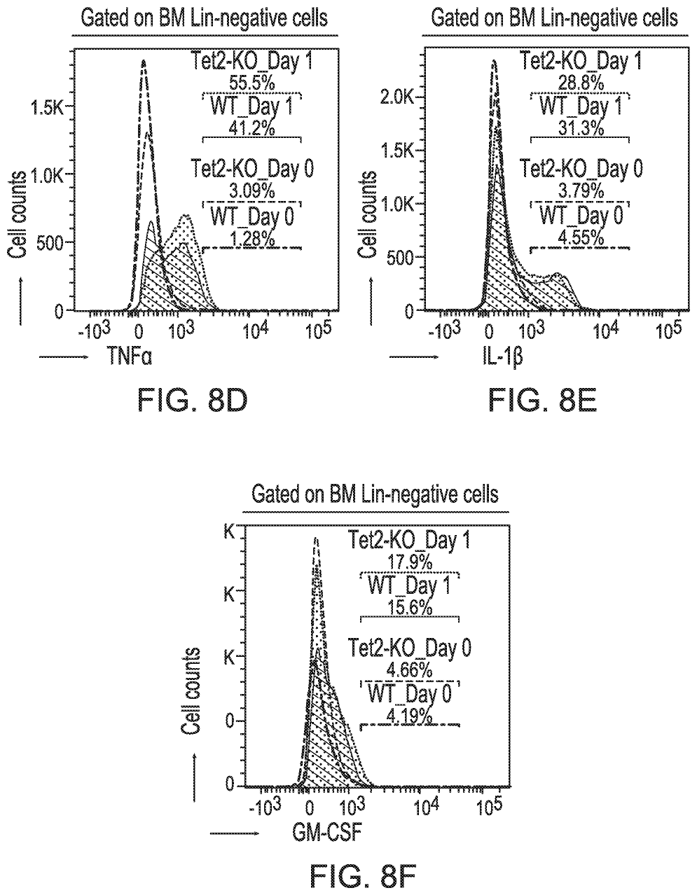

An acute inflammatory challenge can induce an immediate and transient cytokine storm to regulate emergency hematopoiesis and granulopoiesis. Whether inflammation-related cytokines and chemokines are differentially stimulated in LPS-stressed Tet2-KO mice compared to wildtype control mice was next analyzed. Thirty-one cytokines or chemokines were quantified to assess their levels in serum. Fifteen cytokines or chemokines (G-CSF, IL-6, CCL2, CCL4, CXCL1, CCL5, TNFα, CXCL9, CXCL10, IL-10, GM-CSF, IL-1α, IL-1β, M-CSF, IL-2) were found to be stimulated in serum by LPS on Day 1 and Day 2 compared to Day 0 in wildtype or Tet2-KO mice (FIG. 7A). However, it was consistently observed that loss of Tet2 resulted in a profound increase in serum IL-6 levels, not only on Day 0, but a further increase was observed on Day 1 and Day 2 post LPS treatment (FIG. 7A). Ccl2 and Ccl4 were also increased on Day 1 and Day 2 in Tet2-KO mice while TNFα was only elevated on Day 2 in Tet2-KO mice (FIG. 7A). Recent studies have shown that LPS can directly induce IL-6, IL-1β, GM-CSF and TNFα production in HSPCs. Their expression therefore was examined by intracellular staining, flow cytometry and MFI calculation. IL-6 was found to be expressed by more mature bone marrow cells and was also stimulated at an elevated level in immature bone marrow cells (HSPCs) of Tet2-KO mice upon LPS treatment compared to wild type controls (FIGS. 7B-7D). TNFα was found to be expressed at a higher level on Day 1 in Lin-negative bone marrow cells derived from Tet2-KO mice (FIGS. 7D, 8A and 8D). Expression of IL-1α and GM-CSF was stimulated by LPS, but comparable in wild type and Tet2-KO HSPCs (FIGS. 7D, 8B, 8C, 8D and 8F). qRT-PCR assays on Lin-negative bone marrow cells confirmed that IL-6 mRNA was significantly elevated at Day 0, Day 1 and Day 2 post LPS treatment in adult Tet2-KO mice relative to controls (FIG. 7F). Likewise, in juvenile mice, where serum IL-6 and expression of IL-6 were comparable between wildtype and Tet2-KO cells on Day 0, LPS challenge resulted in significantly higher expression of IL-6 in Lin-negative cells derived from Tet2-KO mice relative to controls (FIG. 7G). Interestingly, expression of Ccl2 was also increased in the serum and in Lin-negative cells on Day 1 post LPS treatment in Tet2-KO mice relative to controls (FIGS. 7A, 7F and 7G). Collectively, these results suggest that loss of Tet2 in HSPCs results in elevated IL-6 levels in serum and bone marrow cells under naïve conditions, which is further enhanced upon inflammatory stress. Additional cytokines and chemokines were also elevated in Tet2-KO mice, but not to the same extent as IL-6.

Altered Expression of TLR4/NFκB Pathway Components and a Feed-Forward Loop Involving TLR4/NFκB/IL-6/Morrbid Signaling in the Absence of Tet2.

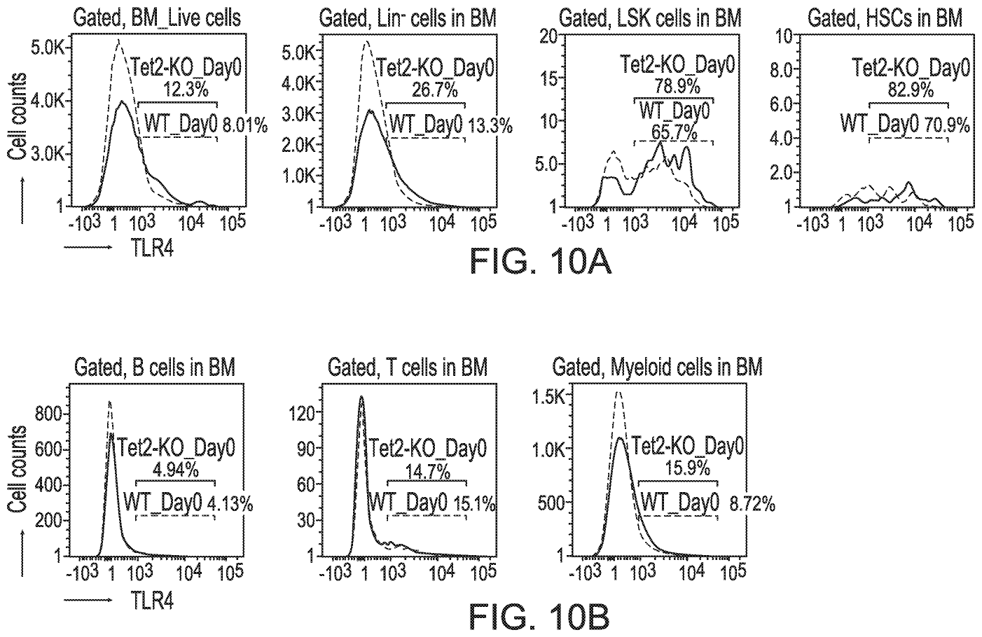

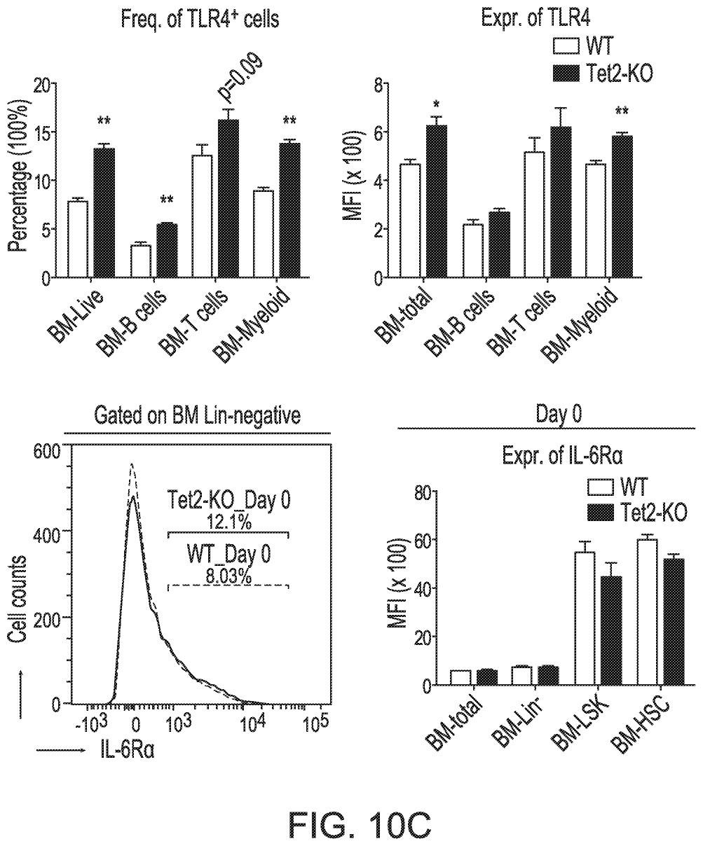

LPS activates canonical TLR4/NFκB signaling, which induces the expression of inflammatory cytokines such as IL-6 to induce emergency hematopoiesis in an effort to resolve infection (a schematic of the signaling pathways is illustrated in FIG. 9A). Without infection induced by exogenous pathogens, TLR4 could be ligated by endogenous ligands such as S100A8 and S100A9 and stimulate a similar innate immune signaling pathway. Consistent with previous studies, it was confirmed that a profound fraction of both LSK cells and HSCs express TLR4 and IL-6 Receptor a (IL-6Rα) by flow cytometry (FIGS. 9B and 10). While the expression of IL-6Rα is comparable between wild type and Tet2-KO naïve mice; frequency of TLR4⁺ cells and expression of TLR4 in Tet2-KO lineage negative cells was significantly increased compared to wild type controls (FIG. 9B). To further evaluate the involvement of TLR4/NFκB and IL-6 signaling in LPS-stressed wild type and Tet2-KO mice, the expression of multiple genes encoding key components of these pathways was analyzed under naïve conditions (Day 0) and after 24 hours (Day 1) or 48 hours (Day 2) post LPS treatment. The expression of Tlr4, Tricam1 (encoding Trif, an intracellular adaptor for TLR4 signaling), Nfkb1 (encoding NFκB1, also known as p50, one of the main subunits of the NFκB family of transcription factors), and Nfkbiz (encoding IκBζ, which binds with NFκB1 for directly regulating the transcription of I16 and Ccl2) was observed in Tet2-KO Lin-negative cells compared to controls (FIG. 9G). Additionally, Apex1 (encoding Apurinic/apyrimidinic (Ap) endonuclease, Ape1, also known as Ref-1, which assists multiple transcription factors including NFκB1 and Stat3 binding to DNA), to be increased in Tet2-KO Lin-cells relative to controls (FIG. 9G). The elevated expression of NFκB1 and phosho-Stat3 in Tet2-KO Lin-negative cells was also validated by flow cytometry and MFI analysis (FIG. 9F). In summary, the gene and protein expression prolife collectively suggest that in addition to elevated expression of IL-6, Tet2-KO Lin-negative cells maintain elevated signaling profile of the TLR4/NFκB pathway under naïve basal conditions as well as upon inflammatory challenge. As NFκB1 and Stat3 are essential transcription factors for regulating innate immune responses, it was hypothesized that the elevated expression of TLR4, IL-6 and Morrbid was likely to be functionally coupled to NFκB1 and Stat3 at the level of transcription in Tet2-KO Lin-negative cells and that a feed forward loop was likely established as a result of Tet2 loss in these cells. Indeed, CHIP-qPCR analysis revealed that IκBζ, a key component of the NFκB complex for modulating DNA binding, was significantly enriched in its binding to the promoter region of both TLR4 and IL-6 genes in Tet2-KO Lin-negative cells compared to controls (FIG. 9H).

APX3330 ((2E)-3-[5-(2,3-dimethoxy-6-methyl-1,4-benzoquinoyl)]-2-propenoic acid) is a well-studied Ape1 redox-signaling inhibitor and has been shown to repress NFκB signaling and the expression of inflammatory cytokines including IL-6 and TNFα as well as impair cancer cell growth. In a CFU assay, treatment of Tet2-KO cells Lin-negative cells with APX3330 resulted in normalization of colony formation in vitro under both primary and secondary plating conditions, which was associated with reduced IKBζ and Stat3 binding to Morrbid promoter in Tet2-KO Lin-negative cells relative to controls (FIGS. 91 & 9J) Similarly, treatment of Tet2-KO Lin-negative cells with a specific and an allosteric inhibitor of SHP2/Shp2, SHP099 (6-(4-amino-4-methyl-1-piperidinyl)-3-(2,3-dichlorophenyl)-2-pyrazinamine), also resulted in decreased expression of phospho-Stat3, CFU formation, IKBζ and Stat3 binding to Morrbid promoter (FIG. 6F, 6H and data not shown).

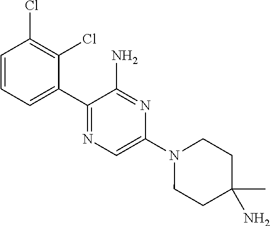 [SMILES: CC1(N)CCN(c2cnc(-c3cccc(Cl)c3Cl)c(N)n2)CC1]

Taken together, these results demonstrate that Tet2 deficiency results in increased expression of TLR4/NFκB/IL-6 signaling components and that both APX3330 and SHP099 are able to block the enhanced colony forming activity of Tet2-KO HSPCs by repressing such signaling.

### Example 2

In this Example, it was analyzed if APX3330 or SHP099 could repress basal inflammation and emergency hematopoiesis in TET2-KO mice.

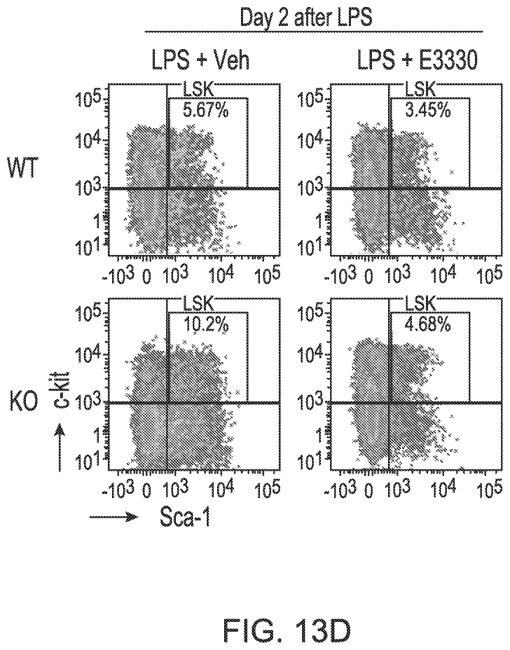

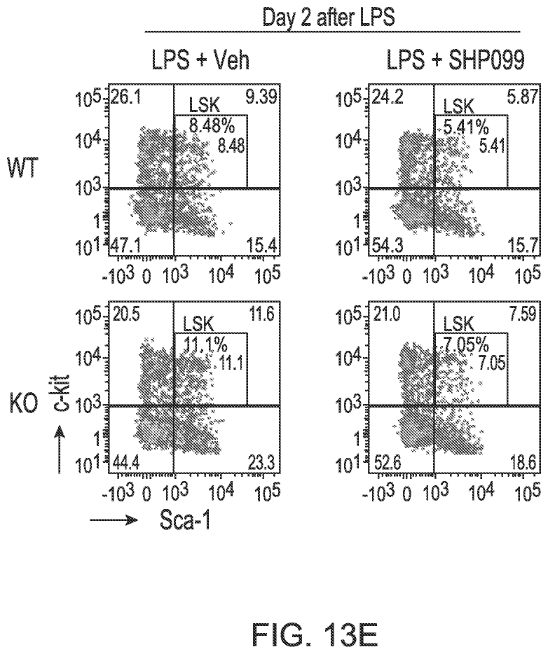

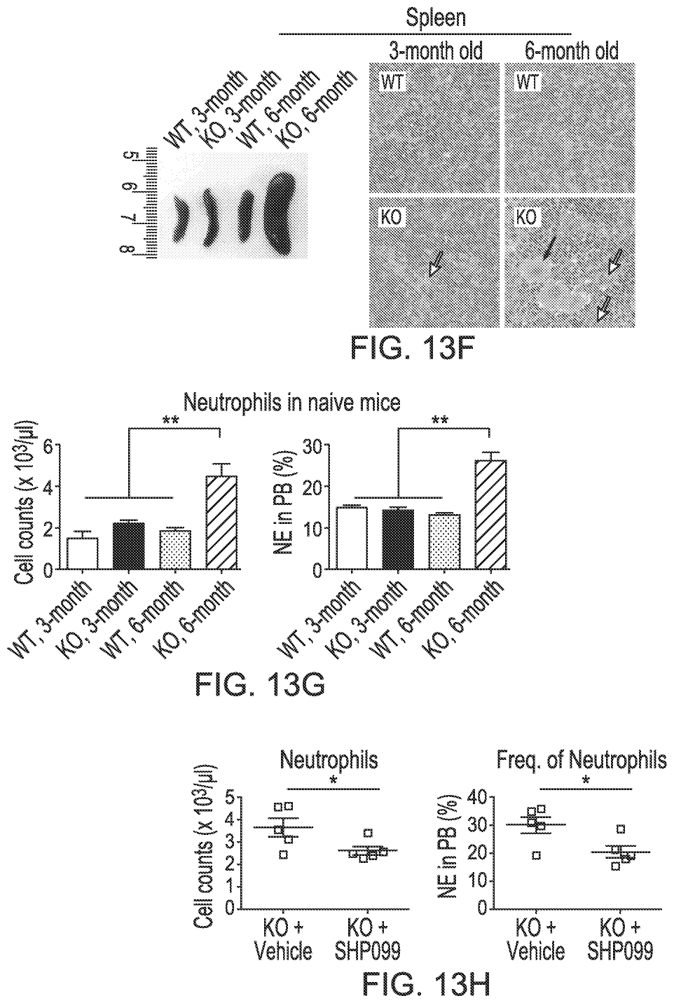

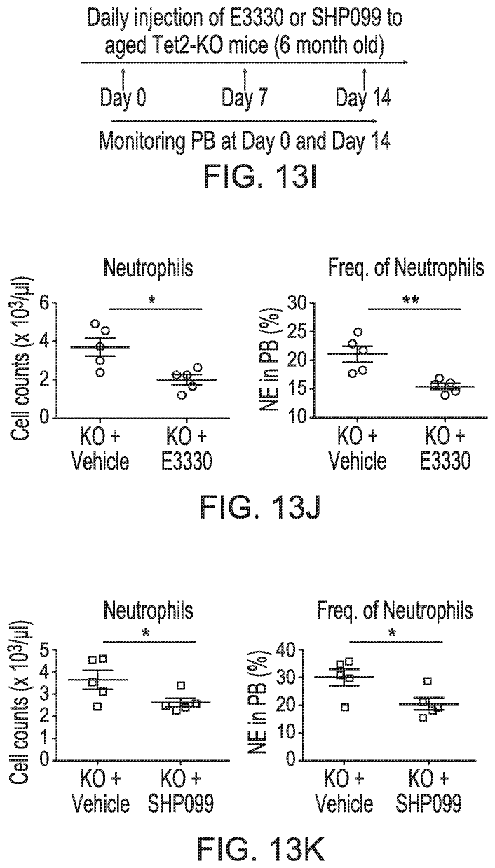

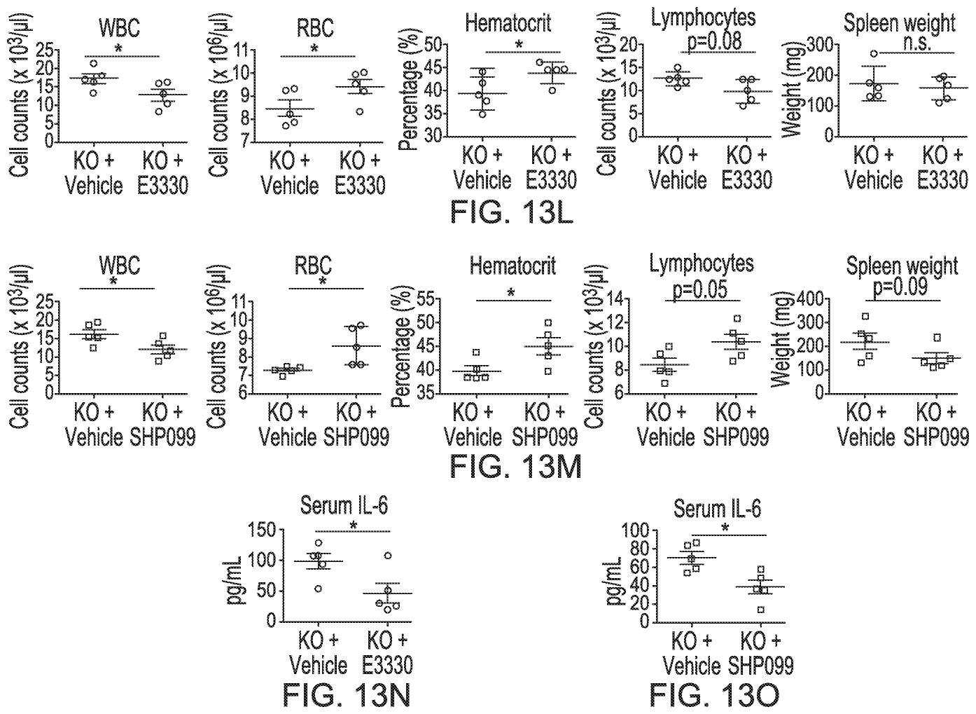

It was next examined if APX3330 or SHP099 could repress inflammation and “emergency hematopoiesis” in Tet2-KO mice in vivo. It was first assessed if APX3330 or SHP099 could normalize LPS-induced acute inflammation. Before challenging the mice with LPS, wildtype and Tet2-KO mice were prophylactically treated with APX3330 or SHP099 for two days. Post LPS treatment, APX3330 or SHP099 was continuously injected in these mice for another two days (FIG. 13A). On day 2, post LPS treatment, mice were sacrificed and analyzed. It was observed that Tet2-KO mice treated with LPS plus APX3330 or LPS plus SHP099 demonstrated a significant correction in the enhanced production of neutrophils and in the expansion of LSK cells compared to mice treated with LPS only (FIGS. 13B-13E). These results demonstrate that APX3330 or SHP099 antagonize LPS-induced acute inflammation, can mediate an in vivo anti-inflammation effect and ameliorate emergency granulopoiesis and emergency hematopoiesis in the absence of Tet2.

In contrast to relatively normal PB hematologic phenotype in 2 to 3 month old Tet2-KO mice, 6-month old Tet2-KO mice manifested more severe signs of MPN including splenomegaly, significantly increased neutrophil counts in PB and significantly increased percentage of neutrophils in WBC (early signs of MPN or CML) compared to wildtype controls (FIGS. 13F and 13G). Furthermore, aged Tet2-KO maintained higher level of serum IL-6 compared to wildtype mice (FIG. 13H). 6-month old Tet2-KO mice were treated with APX3330 or SHP099 to assess the potential therapeutic benefit of these drugs in older mice (daily treatment for 14 days, FIG. 13I). Although, in this short-term treatment regimen, APX3330 and SHP099 failed to reverse the frequency of LSKs and CMPs in Tet2-KO mice, both these drugs were able to significantly reduce the neutrophil, white blood cell (WBC) burden and restored the anemia including the red blood cell (RBC) and platelet counts in Tet2-KO mice (FIGS. 13J-13M). In addition, a reduction in spleen weight was also observed in drug treated Tet2-KO mice, although it did not reach statistical significance. Importantly, Serum IL-6 levels were also reduced upon APX3330 or SHP099 treatment of Tet2-KO mice (FIGS. 13N and 13O). These findings suggest that through targeted inhibition of NFκB/IL-6 signaling axis, short-term treatment with APX3330 or SHP099 offers an anti-inflammatory benefit and leads to the reversal of elevated levels of serum IL-6, neutrophils and onset of anemia in aged Tet2-KO mice.

Based on the foregoing data, Tet2-deficient HSPCs manifest a unique tissue-repair capability in response to inflammatory stress. These findings suggest that Tet2-deficient pre-leukemic HSCs/HPCs are powered with selection advantage. The growth advantage seen in Tet2-deficient pre-LSCs is likely due to elevated NFκB and IL-6 signaling in both mature (supplying IL-6) and immature cells (supplying and responding to IL-6).

IL-6 is one of the major pro-inflammatory cytokines circulating in the blood and also functions locally. In addition to playing an essential role in regulating immunity, IL-6 can also regulate hematopoietic cell development and leukemia transformation. Recent studies utilizing a mouse model of chronic myeloid leukemia (CML) induced by BCR-ABL oncogene mutations showed that leukemia in this model is dependent on increased levels of inflammatory cytokine IL-6. Collectively, along with the reported function of IL-6, the present findings support a hypothesis that increased levels the pro-inflammatory cytokine IL-6 are an essential trigger of MPN or even CML disease observed in Tet2-KO mice with increased grade or incidence with age (FIG. 11A). In addition to IL-6, INFα, INFγ, IL-1α, IL-1β, and TNFα can also directly activate HSCs. As only the level of intracellular IL-6, TNFα, IL-1β and GM-CSF were tested at three defined time points, the possibility that the expression of IL-1β or GM-CSF may have a role in this process cannot be ruled out.

In addition to observing increased levels of IL-6 in Tet2-deficient cells and mice, it was also observed that the expression of multiple components in the TLR4 and IL-6 signaling pathway were upregulated in Tet2-deficient HSPCs, including in LSK cells and HSCs. TLR4 and Sca-1 were among the essential cell-surface proteins that responded to LPS. TLR4 is the main Toll-like receptor specific for LPS and mediates a canonical TLR→NFκB/IκBζ→cytokine signaling pathway. With or without LPS stimulation, it was observed that Tet2-deficient HSPCs exhibited consistently enhanced expression of TLR4, suggesting the possibility that the enhanced sensitivity to LPS in the absence of Tet2 in HSCs may be a result of increased expression of TLR4. This notion is supported by the fact that Tet2 deficient HSCs responded better to LPS stimulation in HSPCs. Interestingly, TRL2 and TRL12 was found to be elevated in its expression in LSK cells in two mouse models of AML respectively. Furthermore, multiple TLRs were found with elevated expression in CD34⁺ progenitor cells from MDS patients.

To further support the observation that TLR4 signaling may be modulated by Tet2 deficiency, it was shown that expression of Nfkb1 and Nfkbiz was upregulated in Tet2-KO mice under basal conditions as well as upon LPS challenge (FIG. 9C). The findings in Tet2-KO lineage negative cells were consistent with previous findings utilizing LPS-treated Tet2-KO macrophages demonstrating enhanced expression of genes in the TLR4/NFκB signaling pathway including Nfkb1, Nfkb2, Nfkbia and Nfkbiz (from their RNA-seq data sheet).

In addition to the altered expression of IL-6 and TLR4, increased Sca-1 expression was consistently observed in Tet2-deficient HSPCs. Sca-1 is an essential cell surface marker for hematopoietic stem cells (FIG. 2A). Loss of Sca-1 in HSCs leads to differentiation defects as well as defects in the repopulating ability of HSCs. Furthermore, loss of Sca-1 also blocks INFα induced emergency hematopoiesis. It was shown that loss of Tet2 resulted in increased expression of Sca-1 and an increase in the fraction of Sca-1⁺ cells in HSPCs (FIGS. 2H, 2I and 9C). With respect to transcription factors (TF) that may possibly regulate Sca-1 expression, Stat1 is a putative candidate. Although increased expression of Stat1 was not observed in Tet2-deficient HSPCs, Stat3 expression was significantly increased on Day 0 (FIG. 9C). Considering that Tet2 is an essential epigenetic regulator, repression of Sca-1 by Tet2 probably occurs via a direct mechanism, similar to the working model saying repression of IL-6 is via a Tet2/HDAC6/IκBζ complex, or through an indirect pathway relying on a long signal transduction such as TLRs→NFkB→IL-6→IL6Rα→Stat1/Stat3 Sca-1.

It has been speculated that acute inflammation or age-related chronic inflammation can induce higher levels of γH2AX, which is used as a sensitive marker for genome stability. In agreement with previous studies, using flow cytometry and MFI analysis, it was validated herein that the alteration of γH2AX was readily detected in a day-to-day comparison between wildtype and Te2-KO cells. Further, MFI value of γH2AX in Tet2-KO cells vs wildtype was higher, indicating a transient higher level of DNA damage in the genome of Tet2-KO hematopoietic progenitor cells (FIGS. 4C & 4D), which supports that Tet2 may have a direct role for surveilling genome stability.

It is generally accepted that initiation and malignancies of cancer, including solid tumor and leukemia, undergo an evolutionary process relying on adaptive advantages of acquired somatic mutations (intrinsic factors) with fitness for niche selection (extrinsic factors), with similar bio-ecological principles as indicated in Darwinian natural selection. Through primary and secondary cBMT assays, it was shown herein that Tet2-deficient HSPCs always outperformed wildtype control cells. Essentially, when wildtype donor cells were isolated from their endogenous microenvironment on Day 2 post LPS stress, they lost their normal repopulating activity (FIGS. 6A-6G). In addition, in agreement with previous findings, even though the primary cBMT only received half of Tet2-deficient bone marrow donors, the recipient animals still developed early signs of MPN or CML, strongly indicating that Tet2-deficiency offers a full-package of growth and repopulating advantage to HSCs.

Based on the results from the Examples, loss of Tet2 results in multiple changes in the level of key proteins including TRL4, IL-6 and Sca-1, which render the self-renewal, differentiation and clonal evolution of mutant HSCs to include myeloid skewing and development of MPN or CML like disease with age.

Given the hyperactivation of the NFκB pathway in Tet2-KO cells, a targeted inhibitor of the Ape1 redox signaling activation of NFκB, APX3330, was examined for impact on emergency hematopoiesis. The results showed that both APX3330 and SHP099 effectively repressed LPS-induced emergency granulopoiesis and LSK expansion. More importantly, the results showed that APX3330 and SHP099 treatment restores the WBC and RBC counts and ratio in aged naïve Tet2-KO mice, suggesting that these drug, through its specific inhibition on Ape1-NFκB or Shp2-Stat3, provides an anti-inflammatory effect in mice bearing AML associated epigenetic mutations often observed in healthy individuals with clonal hematopoiesis. Given that emerging evidence suggests that inflammation very likely play a causative role in the pathology of MPN testing these drug in other pre-leukemic models may be of clinical benefit.

1. A method of slowing the progression of a myeloid malignancy in a subject in need thereof, the method comprising administering an effective amount of 5-(2,3-dimethoxy-6-methyl 1,4-benzoquinoyl)]-2-nonyl-2-propenoic acid (APX3330)) or a pharmaceutically acceptable salt or solvate thereof.

2. The method of claim 1, wherein the APX3330 is a selective inhibitor of the Ref-1 redox function.

3. The method of claim 1 comprising administering from about 10 mg/kg to about 75 mg/kg 5-(2,3-dimethoxy-6-methyl 1,4-benzoquinoyl)]-2-nonyl-2-propenoic acid (APX3330)) or a pharmaceutically acceptable salt or solvate thereof.

4. The method of claim 1, wherein the myeloid malignancy is selected from the group consisting of acute myeloid leukemia (AML), myeloproliferative neoplasm (MPN), myelodysplastic syndrome (MDS) and combinations thereof.

5. The method of claim 1, wherein the myeloid malignancy is acute myeloid leukemia (AML).

6. The method of claim 5 further comprising administering one or more antileukemia chemotherapeutic agent or one or more antileukemia enzyme inhibitor, or a combination thereof.

7. The method of claim 6, wherein the one or more antileukemia chemotherapeutic agent is selected from the group consisting of dexamethasone, vincristine, doxorubicin, and methotrexate.

8. The method of claim 1 further comprising administering one or more carriers, diluents, or excipients, or a combination thereof.

9. The method of claim 1 further comprising administering one or more anti-inflammatory agent.

10. The method of claim 1 further comprising administering SHP099 (6-(4-amino-4-methyl-1-piperidinyl)-3-(2,3-dichlorophenyl)-2-pyrazinamine).

11. A method of inhibiting pre-leukemic stem cell generation in a subject in need thereof, the method comprising administering an effective amount of 5-(2,3-dimethoxy-6-methyl 1,4-benzoquinoyl)]-2-nonyl-2-propenoic acid (APX3330)) or a pharmaceutically acceptable salt or solvate thereof.

12. The method of claim 11, wherein the APX3330 is a selective inhibitor of the Ref-1 redox function.

13. The method of claim 11 comprising administering from about 10 mg/kg to about 75 mg/kg 5-(2,3-dimethoxy-6-methyl 1,4-benzoquinoyl)]-2-nonyl-2-propenoic acid (APX3330)) or a pharmaceutically acceptable salt or solvate thereof.

14. A method of inhibiting production of inflammatory cytokines lacking tet methylcytosine dioxygenase 2 (TET2) in a subject in need thereof, the method comprising administering an effective amount of 5-(2,3-dimethoxy-6-methyl 1,4-benzoquinoyl)]-2-nonyl-2-propenoic acid (APX3330)) or a pharmaceutically acceptable salt or solvate thereof.

15. The method of claim 14, wherein the APX3330 is a selective inhibitor of the Ref-1 redox function.

16. The method of claim 14 comprising administering from about 10 mg/kg to about 75 mg/kg 5-(2,3-dimethoxy-6-methyl 1,4-benzoquinoyl)]-2-nonyl-2-propenoic acid (APX3330)) or a pharmaceutically acceptable salt or solvate thereof.

17. (canceled)

18. (canceled)

19. (canceled)

20. (canceled)

21. The method of claim 11 further comprising administering one or more carriers, diluents, or excipients, or a combination thereof.

22. The method of claim 11 further comprising administering one or more anti-inflammatory agent.

23. The method of claim 14 further comprising administering one or more carriers, diluents, or excipients, or a combination thereof.

24. The method of claim 14 further comprising administering one or more anti-inflammatory agent.


SEQUENCE LISTING (56 sequences)

SEQ ID NO: 1 - DNA, 20 bp, Artificial Sequence
  Synthetic
agtcctggaaatgtgccatc

SEQ ID NO: 2 - DNA, 20 bp, Artificial Sequence
  Synthetic
tcagctccatcagctgaaac

SEQ ID NO: 3 - DNA, 20 bp, Artificial Sequence
  Synthetic
cgcagatcttcaggttcctc

SEQ ID NO: 4 - DNA, 20 bp, Artificial Sequence
  Synthetic
acaaaccccaagtcctcctt

SEQ ID NO: 5 - DNA, 20 bp, Artificial Sequence
  Synthetic
ggtcttcagagacagccagg

SEQ ID NO: 6 - DNA, 20 bp, Artificial Sequence
  Synthetic
gatccaggataacggaggct

SEQ ID NO: 7 - DNA, 20 bp, Artificial Sequence
  Synthetic
tctgagaatgaggggactgg

SEQ ID NO: 8 - DNA, 20 bp, Artificial Sequence
  Synthetic
tgtgctgtgaagatcccaag

SEQ ID NO: 9 - DNA, 19 bp, Artificial Sequence
  Synthetic
gcagcgagcaggagacaga

SEQ ID NO: 10 - DNA, 22 bp, Artificial Sequence
  Synthetic
gctgcttccacaccactgtctt

SEQ ID NO: 11 - DNA, 22 bp, Artificial Sequence
  Synthetic
tgttacttgtagcggcctgttg

SEQ ID NO: 12 - DNA, 21 bp, Artificial Sequence
  Synthetic
ccggagactcagagcaaatcc

SEQ ID NO: 13 - DNA, 23 bp, Artificial Sequence
  Synthetic
tacagcccggtaaaggtatccat

SEQ ID NO: 14 - DNA, 17 bp, Artificial Sequence
  Synthetic
ctggacggccggtcttc

SEQ ID NO: 15 - DNA, 23 bp, Artificial Sequence
  Synthetic
aagttttggtccacagcctattt

SEQ ID NO: 16 - DNA, 19 bp, Artificial Sequence
  Synthetic
cggtgcataagcaacagcc

SEQ ID NO: 17 - DNA, 20 bp, Artificial Sequence
  Synthetic
gcctccgaaaaacttggacc

SEQ ID NO: 18 - DNA, 23 bp, Artificial Sequence
  Synthetic
ggctcttctacctctagcaaaag

SEQ ID NO: 19 - DNA, 21 bp, Artificial Sequence
  Synthetic
taatggcctggagtcctacac

SEQ ID NO: 20 - DNA, 22 bp, Artificial Sequence
  Synthetic
ctcttggagtaaaatgtgccca

SEQ ID NO: 21 - DNA, 23 bp, Artificial Sequence
  Synthetic
gacttgcggcaatacatgttttc

SEQ ID NO: 22 - DNA, 24 bp, Artificial Sequence
  Synthetic
aagctcatggaatcaatcagatca

SEQ ID NO: 23 - DNA, 20 bp, Artificial Sequence
  Synthetic
ttaagcattcctcccaagca

SEQ ID NO: 24 - DNA, 20 bp, Artificial Sequence
  Synthetic
gcaagcatggttctttgtga

SEQ ID NO: 25 - DNA, 20 bp, Artificial Sequence
  Synthetic
agttgccttcttgggactga

SEQ ID NO: 26 - DNA, 20 bp, Artificial Sequence
  Synthetic
tccacgatttcccagagaac

SEQ ID NO: 27 - DNA, 20 bp, Artificial Sequence
  Synthetic
agggtctgggccatagaact

SEQ ID NO: 28 - DNA, 21 bp, Artificial Sequence
  Synthetic
ccaccacgctcttctgtctac

SEQ ID NO: 29 - DNA, 20 bp, Artificial Sequence
  Synthetic
attgggatcatcttgctggt

SEQ ID NO: 30 - DNA, 19 bp, Artificial Sequence
  Synthetic
cctgctgttcacagttgcc

SEQ ID NO: 31 - DNA, 20 bp, Artificial Sequence
  Synthetic
gaaacagcaggaagtgggag

SEQ ID NO: 32 - DNA, 20 bp, Artificial Sequence
  Synthetic
catgaagctctgcgtgtctg

SEQ ID NO: 33 - DNA, 20 bp, Artificial Sequence
  Synthetic
tgtcatcagggactttgctg

SEQ ID NO: 34 - DNA, 20 bp, Artificial Sequence
  Synthetic
tgttcttctcctgcctgaca

SEQ ID NO: 35 - DNA, 20 bp, Artificial Sequence
  Synthetic
tgtccagcggtgtgttacat

SEQ ID NO: 36 - DNA, 20 bp, Artificial Sequence
  Synthetic
cagccacctagagatcagcc

SEQ ID NO: 37 - DNA, 20 bp, Artificial Sequence
  Synthetic
gaacgataacctttgcaggc

SEQ ID NO: 38 - DNA, 18 bp, Artificial Sequence
  Synthetic
catcacacggagggcttc

SEQ ID NO: 39 - DNA, 20 bp, Artificial Sequence
  Synthetic
tatcgggtgacacagttgga

SEQ ID NO: 40 - DNA, 20 bp, Artificial Sequence
  Synthetic
tgaatggacttccccttcag

SEQ ID NO: 41 - DNA, 20 bp, Artificial Sequence
  Synthetic
agcacttggtctcttggagg

SEQ ID NO: 42 - DNA, 20 bp, Artificial Sequence
  Synthetic
gcaaatctgccacactcaag

SEQ ID NO: 43 - DNA, 20 bp, Artificial Sequence
  Synthetic
ctgaatatttccctcctggg

SEQ ID NO: 44 - DNA, 21 bp, Artificial Sequence
  Synthetic
tcccgtacagatgtccatgat

SEQ ID NO: 45 - DNA, 20 bp, Artificial Sequence
  Synthetic
ctgctccaggtagcgtgtgt

SEQ ID NO: 46 - DNA, 18 bp, Artificial Sequence
  Synthetic
ctcagccccggagacagt

SEQ ID NO: 47 - DNA, 20 bp, Artificial Sequence
  Synthetic
ggcagatgggtaagcaaaga

SEQ ID NO: 48 - DNA, 20 bp, Artificial Sequence
  Synthetic
caattacctgcccctaccct

SEQ ID NO: 49 - DNA, 22 bp, Artificial Sequence
  Synthetic
gacggccaggtcatcactattg

SEQ ID NO: 50 - DNA, 20 bp, Artificial Sequence
  Synthetic
aggaaggctggaaaagagcc

SEQ ID NO: 51 - DNA, 30 bp, Artificial Sequence
  Synthetic
cctgcgtttaaataacatcagctttagctt

SEQ ID NO: 52 - DNA, 28 bp, Artificial Sequence
  Synthetic
gcacaatgtgacgtcgtttagcatcgaa

SEQ ID NO: 53 - DNA, 20 bp, Artificial Sequence
  Synthetic
cacaagacacggcaactgat

SEQ ID NO: 54 - DNA, 20 bp, Artificial Sequence
  Synthetic
tcgcaggagggaagttagaa

SEQ ID NO: 55 - DNA, 20 bp, Artificial Sequence
  Synthetic
agcacgagtcatctggttcc

SEQ ID NO: 56 - DNA, 20 bp, Artificial Sequence
  Synthetic
acccagtcccctcattctca